(19) United States
(12) Patent Application Publication
(10) Pub. No.: US 2021/0008371 A1
(43) Pub. Date: Jan. 14, 2021

(54) PHYSIOLOGICALLY-GUIDED NEUROMODULATION THERAPY

(71) Applicant: Boston Scientific Neuromodulation Corporation, Valencia, CA (US)

(72) Inventor: Luca Antonello Annecchino, London (GB)

(21) Appl. No.: 16/910,916

(22) Filed: Jun. 24, 2020

Related U.S. Application Data
(60) Provisional application No. 62/872,576, filed on Jul. 10, 2019.

(51) Int. Cl. A61N 1/36; G16H 50/30

(52) U.S. Cl. CPC A61N 1/36071; G16H 50/30; A61N 1/36132; A61N 1/36135

(57) ABSTRACT

Systems and methods for optimizing neuromodulation field design for pain therapy are discussed. An exemplary neuromodulation system includes an electrostimulator to stimulate a target neural element with first neuromodulation energy, a data receiver to receive pain data including pain sites experiencing pain, and to physiological data including body sites responsive to the first neuromodulation energy. A processor circuit can determine a pain distribution and a response distribution over respective sets of dermatomal compartments, generate a pain targeting metric (PTM) using the pain distribution and the response distribution, and determine an optimal stimulation setting for neuromodulation pain therapy based on the pain targeting metric.

### CLAIM OF PRIORITY

This application claims the benefit of priority under 35 U.S.C. § 119(e) of U.S. Provisional Patent Application Ser. No. 62/872,576, filed on Jul. 10, 2019, which is herein incorporated by reference in its entirety.

### TECHNICAL FIELD

This document relates generally to medical devices, and more particularly, to systems, devices and methods for delivering neuromodulation.

### BACKGROUND

Neuromodulation (or “neural neuromodulation”, also referred to as “neurostimulation” or “neural stimulation”) has been proposed as a therapy for a number of conditions. Often, neuromodulation and neural stimulation may be used interchangeably to describe excitatory stimulation that causes action potentials as well as inhibitory and other effects. Examples of neuromodulation include Spinal Cord Stimulation (SCS), Deep Brain Stimulation (DBS), Peripheral Nerve Stimulation (PNS), and Functional Electrical Stimulation (FES). SCS, by way of example and not limitation, has been used to treat chronic pain syndromes.

Conventional SCS delivers electrical pulses to the dorsal column fibers in the dorsal aspect of the spinal cord, which in turn activate a set of inhibitory neurons in the dorsal horn, thereby masking the transmission of pain signals from the periphery of the body to the brain. Notably, the dorsal column fibers are organized in a spatially-dependent manner according to the region of the body with which they respectively interface. Accordingly, it is desirable to optimally target the neuromodulation to the precise fibers that correspond to the source of pain to be treated, while minimizing stimulation of other fibers in order to reduce or avoid side effects.

However, it can be a challenge to find a desirable or optimal location (a sweet spot) for the neuromodulation field during programming of a neuromodulation device. Optimal-location searching involves a healthcare professional adjusting the targeting of the neuromodulation to provide optimal pain relief for the patient with minimal discomfort. State-of-the-art systems provide the ability to create complex electrode configurations to create virtual poles by combining fractionalized current output from physical electrodes in the vicinity of the desired virtual pole. Though beneficial for targeting specific areas, the complexity of adjusting multiple virtual poles to provide optimal neuromodulation for the patient presents a number of problems including extended duration of testing; and the possibility of causing patient discomfort during testing. Complex neuroanatomy in certain spinal cord regions also increases the difficulty in finding an optimal neuromodulation field design and programming the neuromodulation device accordingly to achieve desired therapeutic effect of pain relief.

### SUMMARY

The following examples illustrate various aspects of the embodiments described herein.

Example 1 is a system for controlling neuromodulation therapy for pain relief in a patient. The system comprises an electrostimulator, a data receiver, and a processor circuit. The electrostimulator can be configured to apply first neuromodulation energy to a target neural element of the patient according to a stimulation setting. The data receiver can be configured to receive pain data including information of a pain site on a body of the patient, and to receive physiological data including information of patient body site responsive to the applied first neuromodulation energy. The processor circuit can be configured to: determine a pain distribution across a first set of dermatomal compartments using the received pain data, and determine a response distribution across a second set of dermatomal compartments using the received physiological data; generate a pain targeting metric (PTM) using the pain distribution and the response distribution, the PTM representing a spatial correspondence between the pain site and the body site responsive to the applied first neuromodulation energy at one or more dermatomal compartments; and determine an optimal stimulation setting for neuromodulation pain therapy using the generated PTM.

In Example 2, the subject matter of Example 1 optionally includes the electrostimulator that can be configured to apply second neuromodulation energy to the target neural element for pain relief in accordance with the optimal stimulation setting, the second neuromodulation energy different from the first neuromodulation energy.

In Example 3, the subject matter of any one or more of Examples 1-2 optionally includes the received pain data that can include a pain drawing of the pain site associated with a pain score, and the received physiological data can include a response map of the body site responsive to the applied first neuromodulation energy, the body site associated with a response score.

In Example 4, the subject matter of Example 3 optionally includes the response map that can include a paresthesia drawing of body site experiencing paresthesia in response to the applied first neuromodulation energy.

In Example 5, the subject matter of any one or more of Examples 3-4 optionally includes at least one sensor configured to sense a physiological response to the applied first neuromodulation energy, wherein the response map can include the sensed physiological response at the body site of the patient.

In Example 6, the subject matter of Example 5 optionally includes the at least one sensor that can include: at least one electromyography (EMG) sensor; at least one electrospinogram (ESG) sensor; at least one electrically evoked compound action potential (eCAP) sensor; at least one impedance sensor; at least one photoplethysmography (PPG) sensor; at least one near-infrared spectroscopy (NIRS) sensor; at least one doppler flowmetry sensor; at least one accelerometer sensor; or at least one gyroscope sensor.

In Example 7, the subject matter of any one or more of Examples 3-6 optionally includes the processor circuit that can be configured to: pixelate the pain drawing into pixels corresponding to anatomical point locations of the pain site, determine for each of the first set of dermatomal compartments a respective derma tore-level pain effect using the pixelated pain drawing, and generate the pain distribution using the dermatome-level pain effects of the first set of dermatomal compartments; pixelate the response map into pixels corresponding to anatomical point locations on the body site responsive to the applied first neuromodulation energy, determine for each of the second set of dermatomal compartments a respective dermatome-level physiological response using the pixelated response map, and generate the response distribution using the dermatome-level physiological responses of the second set of dermatomal compartments.

In Example 8, the subject matter of Example 7 optionally includes the processor circuit that can be configured to: generate a first dermatomal metric from the pain distribution, and generate a second dermatomal metric from the response distribution; and generate the PTM using the first and second dermatomal metrics.

In Example 9, the subject matter of Example 8 optionally includes the first dermatomal metric that can include a center of mass of the pixelated pain drawing based on the dermatome-level pain effects of the first set of dermatomal compartments, and the second dermatomal metric that can include a center of mass of the pixelated response map based on the dermatome-level physiological responses of the second set of dermatomal compartments.

In Example 10, the subject matter of any one or more of Examples 8-9 optionally includes the first dermatomal metric that can include a peak dermatome representing a dermatomal compartment having a largest dermatome-level pain effect among the first set of dermatomal compartments, and the second dermatomal metric that can include a peak dermatome representing a dermatomal compartment having a largest dermatome-level physiological response among the second set of dermatomal compartments.

In Example 11, the subject matter of any one or more of Examples 8-10 optionally includes the first dermatomal metric that can include a dermatomal spread representing a subset of the first set of dermatomal compartments that have a dominant dermatome-level pain effect, and the second dermatomal metric that can include a dermatomal spread representing a subset of the second set of dermatomal compartments that have a dominant dermatome-level physiological response.

In Example 12, the subject matter of any one or more of Examples 7-11 optionally includes the processor circuit that can be configured to generate the PTM using an overlap between the pixelated pain drawing and the pixelated response map. The overlap can be based on the dermatome-level pain effects and the dermatome-level physiological responses across a union of the first and second sets of dermatomal compartments.

In Example 13, the subject matter of Example 12 optionally includes the processor circuit that can be configured to generate the PTM further using at least one of: a ratio of the overlap to a sum of the dermatome-level pain effects across the first set of dermatomal compartments; or a ratio of the overlap to a sum of the dermatome-level physiological responses across the second set of dermatomal compartments.

In Example 14, the subject matter of any one or more of Examples 1-13 optionally includes the processor circuit that can be configured to evaluate the PTM for each of a plurality of candidate stimulation settings, and to select from the plurality of candidate stimulation settings an optimal stimulation setting with a corresponding VIM satisfying a specific condition.

In Example 15, the subject matter of any one or more of Examples 1-14 optionally includes a user interface that can be configured to display one of more of the pain distribution, the response distribution, or the PTM, and to receive a user input for adjusting one or more stimulation parameters including: a stimulation electrode position; a stimulation pulse width; a stimulation amplitude; a stimulation rate; or a stimulation pulse waveform.

Example 16 is a method for controlling neuromodulation therapy for pain relief in a patient. The method comprises steps of: receiving pain data including information of a pain site on a body of the patient; applying first neuromodulation energy to a target neural element of the patient according to a stimulation setting; receiving physiological data including information of a body site responsive to the applied first neuromodulation energy; determining a pain distribution across a first set of dermatomal compartments using the received pain data, and determining a response distribution across a second set of dermatomal compartments using the received physiological data; generate a pain targeting metric (PTM) using the pain distribution and the response distribution, the PTM representing a spatial correspondence between the pain site and the body site responsive to the applied first neuromodulation energy at one or more dermatomal compartments; and determining an optimal stimulation setting for neuromodulation pain therapy using the generated PTM.

In Example 17, the subject matter of Example 16 optionally includes the received pain data that can include a pain drawing and the received physiologic data that can include a response map. The step of determining the pain distribution can include steps of: pixelating the pain drawing; determining for each of the first set of dermatomal compartments a respective dermatome-level pain effect using the pixelated pain drawing; and generating the pain distribution using the dermatome-level pain effects of the first set of dermatomal compartments. The step of determining the response distribution can include steps of: pixelating the response map; determining for each of the second set of dermatomal compartments a respective dermatome-level physiological response using the pixelated response map; and generating the response distribution using the dermatome-level physiological responses of the second set of dermatomal compartments.

In Example 18, the subject matter of Example 17 optionally includes generating the PTM using a comparison between a first dermatomal metric derived from the pain distribution and a second dermatomal metric derived from the response distribution.

In Example 19, the subject matter of Example 18 optionally includes the first dermatomal metric that can include a center of mass of the pixelated pain drawing based on the dermatome-level pain effects of the first set of dermatomal compartments, and the second dermatomal metric that can include a center of mass of the pixelated response map based on the dermatome-level physiological responses of the second set of dermatomal compartments. The first dermatomal metric can include a peak dermatome representing a dermatomal compartment having a largest dermatome-level pain effect among the first set of dermatomal compartments, and the second dermatomal metric can include a peak dermatome representing a dermatomal compartment having a largest dermatome-level physiological response among the second set of dermatomal compartments. The first dermatomal metric can include a dermatomal spread representing a subset of the first set of dermatomal compartments that have a dominant dermatome-level pain effect, and the second dermatomal metric can include a dermatomal spread representing a subset of the second set of dermatomal compartments that have a dominant dermatome-level physiological response.

In Example 20, the subject matter of any one or more of Examples 17-19 optionally includes generating the PTM using at least one of: an overlap between the pixelated pain drawing and the pixelated response map, the overlap based on the dermatome-level pain effects and the dermatome-level physiological responses across a union of the first and second sets of dermatomal compartments; a ratio of the overlap to a sum of the dermatome-level pain effects across the first set of dermatomal compartments; or a ratio of the overlap to a sum of the dermatome-level physiological responses across the second set of dermatomal compartments.

In Example 21, the subject matter of any one or more of Examples 16-20 optionally includes evaluating the PTM for each of a plurality of candidate stimulation settings, and selecting from the plurality of candidate stimulation settings an optimal stimulation setting with a corresponding PTM satisfying a specific condition.

In Example 22, the subject matter of any one or more of Examples 16-21 optionally include applying second neuromodulation energy to the target neural element for pain relief in accordance with the optimal stimulation setting. The second neuromodulation energy can be different from the first neuromodulation energy.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates a portion of a spinal cord.

FIG. 2 illustrates, by way of example, an embodiment of a neuromodulation system.

FIG. 3 illustrates, by way of example, an embodiment of a neuromodulation device, such as may be implemented in the neuromodulation system of FIG. 2.

FIG. 4 illustrates, by way of example, an embodiment of a programming device, such as may be implemented as the programming device in the neuromodulation system of FIG. 2.

FIG. 5 illustrates, by way of example, an implantable neuromodulation system and portions of an environment in which system may be used.

FIG. 6 illustrates, by way of example, an embodiment of a Spinal Cord Stimulation (SCS) system, which also may be referred to as a Spinal Cord Neuromodulation (SCUM) system.

FIG. 7 illustrates, by way of example, some features of the neuromodulation leads and a pulse generator.

FIGS. 8-9 are schematic views of embodiments of neuromodulation lead placement on a patient's spinal cord.

FIG. 10 illustrates a schematic view of the electrical neuromodulation lead showing an example of the fractionalization of the anodic current delivered to the electrodes on the electrical neuromodulation lead.

FIGS. 11A-11B illustrate, by way of example and not limitation, electrode arrangements and test regions of neural tissue along the electrode arrangements.

FIGS. 12A-12C illustrate, by way of example and not limitation, neural tissue locations that may be targeted within the test region in one, two and three dimensions, respectively.

FIG. 13 illustrates an example of a method for finding a sweet spot for sub-perception neuromodulation.

FIG. 14 illustrates, by way of example, aspects of a binary search routine as a rostrocaudal focus routine.

FIG. 15 illustrates an example of the binary search routine.

FIGS. 16A-16C illustrate, by way of example, an edge search routine.

FIG. 17 is a diagram illustrating a programming device in greater detail according to an embodiment.

FIGS. 18 and 19 are diagrams illustrating, by way of example and not limitation, anatomical regions on a patient body where pain is felt, or anatomical region on a patient body where a neurostimulation effect such as paresthesia is perceived.

FIG. 20 is a block diagram illustrating, by way of example and not limitation, a neuromodulation system configured to optimize electrostimulation at a neural element for pain control.

FIG. 21 is a block diagram illustrating, by way of example and not limitation, a dermatomal coverage analyzer circuit configured to generate a pain targeting metric (PTM).

FIG. 22 is a diagram illustrating, by way of example and not limitation, a pain distribution, and a plurality of paresthesia distributions corresponding to different stimulation fields, across multiple dermatomal compartments.

FIGS. 23A-23C are diagrams illustrating, by way of example and not limitation, dermatomal metrics and PTMs based on the pain distribution and the paresthesia distributions.

FIG. 24 is a diagram illustrating, by way of example and not limitation, a portion of a user interface for performing a pain-targeting test to find an optimal parameter setting.

FIG. 25 is a flow chart illustrating, by way of example and not limitation, a method for optimizing neurostimulation of a neural element for pain relief.

FIG. 26 illustrates generally a block diagram of an example machine 2500 upon which any one or more of the techniques (e.g., methodologies) discussed herein may perform.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Various embodiments described herein involve neuromodulation of target neural element such as a portion of a spinal cord to relieve pain. The neuromodulation may include sub-perception stimulation therapeutically effective but non-perceptible (apart from any therapeutic effects) to the patient. Thus, the therapeutic effects of the sub-perception neuromodulation can be perceived by the patient.

The complex spinal cord structure resides in a complex three-dimensional environment. For example, the thickness of the cerebrospinal fluid (CSF), which is between the spinal cord and the epidural space, varies along the spine. Thus, the distance between the spinal cord and one or more neuromodulation leads within the epidural space likely varies. Furthermore, neither the leads nor the spinal cord form simple straight lines. The positions of implanted neuromodulation leads can also vary and are not perfectly parallel to the spinal cord. Additionally, the neuroanatomy of the spinal cord region can vary from patient to patient. It is desirable to program a modulation system to account for electrode positions and variations in conductance through the electrodes and tissue to improve therapy programming.

Various embodiments of the present subject matter provide systems and methods for facilitating the creation of a target neuromodulation field with improved precision using a dermatomal coverage of pain area, thereby easing the burden of searching for a sweet spot for spinal cord stimulation (SCS) for pain relief. The dermatomal coverage may be represented by a spatial correspondence between patient body sites of pain perception and patient body sites of responsive to diagnostic stimulation (in contrast to therapeutic stimulation for pain relief) over one or more dermatomal compartments. An exemplary neuromodulation system determines a pain distribution across a first set of dermatomal compartments using pain data (e.g., a pain drawing), and to determine a response distribution across a second set of dermatomal compartments using patient physiological data responsive to diagnostic stimulation (e.g., a paresthesia drawing or a sensor data map). One or more pain targeting metrics (PTM) may be generated based on the pain distribution and the response distribution. Based on the PTM, the neuromodulation system may determine an optimal stimulation setting for use in neuromodulation pain therapy.

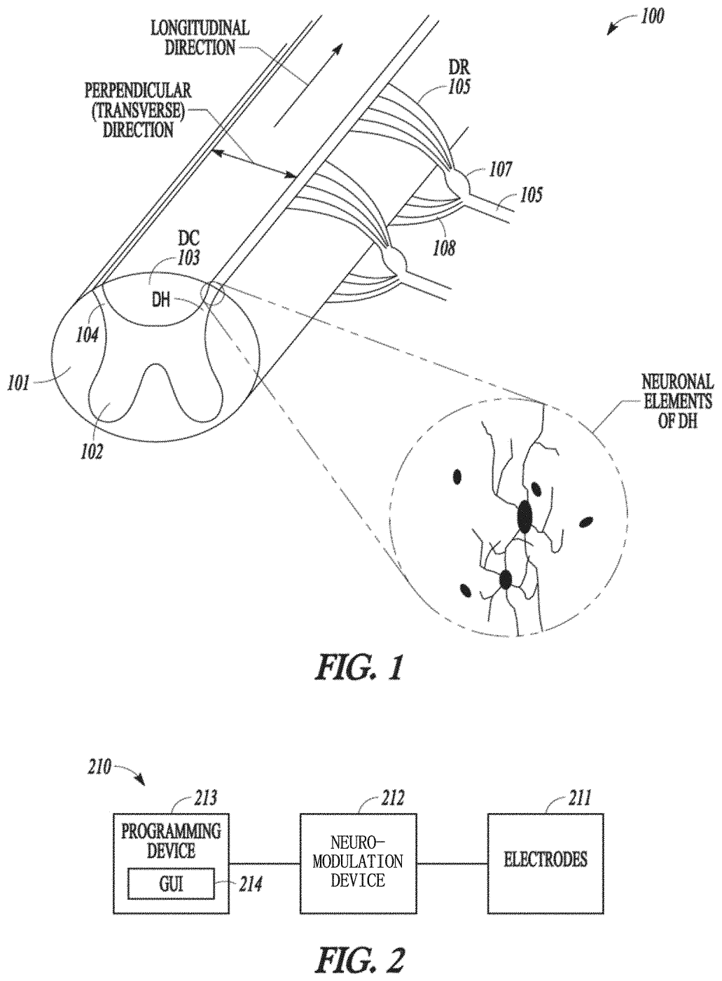

As some embodiments described herein involve SCS (also referred to as spinal cord neuromodulation), a brief description of the physiology of the spinal cord is provided herein to assist the reader. FIG. 1 illustrates, by way of example, a portion of a spinal cord 100 including white matter 101 and gray matter 102 of the spinal cord. The gray matter 102 includes cell bodies, synapse, dendrites, and axon terminals. Thus, synapses are located in the gray matter. White matter 101 includes myelinated axons that connect gray matter areas. A typical transverse section of the spinal cord includes a central “butterfly” shaped central area of gray matter 102 substantially surrounded by an ellipse-shaped outer area of white matter 101. The white matter of the dorsal column (DC) 103 includes mostly large myelinated axons that form afferent fibers that run in an axial direction. The dorsal portions of the “butterfly” shaped central area of gray matter are referred to as dorsal horns (DH) 104. In contrast to the DC fibers that run in an axial direction, DH fibers can be oriented in many directions, including perpendicular to the longitudinal axis of the spinal cord. Examples of spinal nerves 105 are also illustrated, including a dorsal root (DR) 105, dorsal root ganglion 107 and ventral root 108. The dorsal root 105 mostly carries sensory signals into the spinal cord, and the ventral root functions as an efferent motor root. The dorsal and ventral roots join to form mixed spinal nerves 105.

SCS has been used to alleviate pain. A therapeutic goal for conventional SCS programming has been to maximize stimulation (i.e., recruitment) of the DC fibers that run in the white matter along the longitudinal axis of the spinal cord and minimal stimulation of other fibers that run perpendicular to the longitudinal axis of the spinal cord (dorsal root fibers, predominantly), as illustrated in FIG. 1. The white matter of the DC includes mostly large myelinated axons that form afferent fibers. While the full mechanisms of pain relief are not well understood, it is believed that the perception of pain signals is inhibited via the gate control theory of pain, which suggests that enhanced activity of innocuous touch or pressure afferents via electrical stimulation creates interneuronal activity within the DH of the spinal cord that releases inhibitory neurotransmitters (Gamma-Aminobutyric Acid (GABA), glycine which in turn, reduces the hypersensitivity of wide dynamic range (WDR) sensory neurons to noxious afferent input of pain signals traveling from the dorsal root (DR) neural fibers that innervate the pain region of the patient, as well as treating general WDR ectopy. Consequently, the large sensory afferents of the DC nerve fibers have been targeted for stimulation at an amplitude that provides pain relief. Current implantable neuromodulation systems typically include electrodes implanted adjacent, i.e., resting near, or upon the dura, to the dorsal column of the spinal cord of the patient and along a longitudinal axis of the spinal cord of the patient.

Activation of large sensory DC nerve fibers also typically creates the paresthesia sensation that often accompanies conventional SCS therapy. Although alternative or artifactual sensations, such as paresthesia, are usually tolerated relative to the sensation of pain, patients sometimes report these sensations to be uncomfortable, and therefore, they can be considered an adverse side-effect to neuromodulation therapy in some cases. Some embodiments discussed herein deliver sub-perception therapy that is therapeutically effective to treat pain, for example. However, the patient does not sense the delivery of the neuromodulation field (e.g. paresthesia) during a sub-perception therapy. Sub-perception therapy may be provided using higher frequency neuromodulation (e.g. about 1500 Hz or above) of the spinal cord. Sub-perception neuromodulation may also be provided through neuromodulation field shaping (e.g., using multiple independent current control, or MICC), and temporal shaping of pulse train (e.g., burst, longer pulses). It appears that these higher frequencies may effectively block the transmission of pain signals in the afferent fibers in the DC. Some embodiments herein selectively modulate DH tissue or DR tissue over DC tissue to provide sub-perception therapy.

Such selective neuromodulation may be delivered at lower frequencies. For example, the selective neuromodulation may be delivered at frequencies less than 1,200 Hz. The selective neuromodulation may be delivered at frequencies less than 1,000 Hz in some embodiments. In some embodiments, the selective neuromodulation may be delivered at frequencies less than 500 Hz. In some embodiments, the selective neuromodulation may be delivered at frequencies less than 350 Hz. In some embodiments, the selective neuromodulation may be delivered at frequencies less than 130 Hz. The selective neuromodulation may be delivered at low frequencies (e.g. as low as 2 Hz). The selective neuromodulation may be delivered even without pulses (e.g. 0 Hz) to modulate some neural tissue. By way of example and not limitation, the selective neuromodulation may be delivered within a frequency range selected from the following frequency ranges: 2 Hz to 1,200 Hz; 2 Hz to 1,000 Hz, 2 Hz to 500 Hz; 2 Hz to 350 Hz; or 2 Hz to 130 Hz. Systems may be developed to raise the lower end of any these ranges from 2 Hz to other frequencies such as, by way of example and not limitation, 10 Hz, 20 Hz, 50 Hz or 100 Hz.

The selective neuromodulation may be delivered with a duty cycle, in which stimulation (e.g. a train of pulses) is delivered during a Stimulation ON portion of the duty cycle, and is not delivered during a Stimulation OFF portion of the duty cycle. The selected modulation may be delivered with fixed or variable pulse widths. By way of example and not limitation, the duty cycle may be about 10%±5%, 20%±5%, 30%±5%, 40%±5%, 50%±5% or 60%±5%. For example, a burst of pulses for 10 ins during a Stimulation ON portion followed by 15 ms without pulses corresponds to a 40% duty cycle.

While SCS is specifically discussed as a neuromodulation therapy, such discussion is by way of example and not limitation. Various embodiments of pain targeting and dermatomal coverage analysis as discussed in this document may be applied to other neuromodulation therapies, such as Peripheral Nerve Stimulation (PNS) therapies to alleviate pain.

FIG. 2 illustrates an embodiment of a neuromodulation system 210, which includes electrodes 211, a neuromodulation device 212, and a programming device 213. The electrodes 211 are configured to be placed on or near one or more neural targets in a patient. The electrodes 211 may form part of an electrode arrangement. The neuromodulation device 212 is configured to be electrically connected to electrodes 211 and deliver neuromodulation energy, such as in the form of electrical pulses, to the one or more neural targets though electrodes 211. The delivery of the neuromodulation is controlled using a plurality of neuromodulation parameters, such as neuromodulation parameters specifying the electrical pulses and a selection of electrodes through which each of the electrical pulses is delivered. In various embodiments, at least some parameters of the plurality of neuromodulation parameters are programmable, either manually by a user (e.g., physician or other caregiver) or at least partially automatically in a closed-loop neuromodulation system. The programming device 213 may provide the user with accessibility to the user-programmable parameters. In various embodiments, the programming device 213 is configured to be communicatively coupled to neuromodulation device via a wired or wireless link. In various embodiments, the programming device 213 includes a graphical user interface (GUI) 214 that allows the user to set and/or adjust values of the user-programmable neuromodulation parameters.

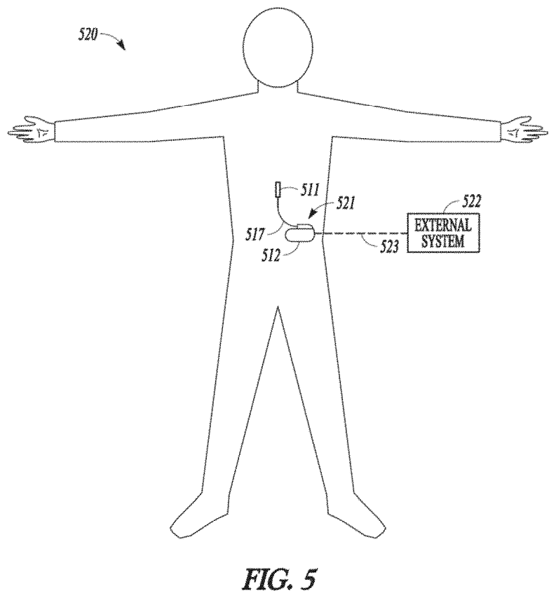

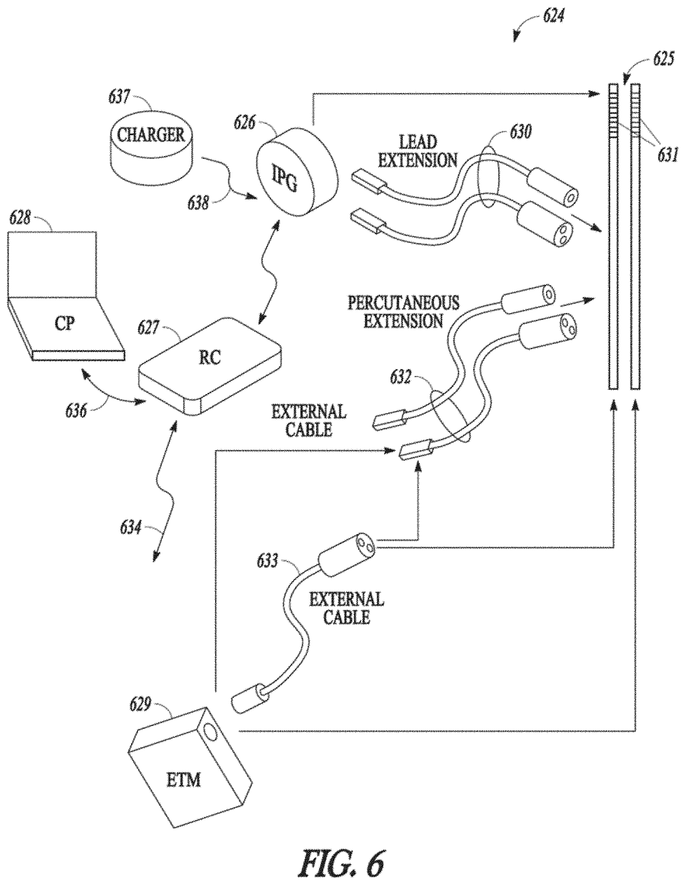

In various embodiments, the neuromodulation system 210 can include implantable and external elements. For example, the neuromodulation device 212 can be an implantable neuromodulation device, the electrodes 211 can include electrodes in one or more implantable lead and/or the implantable neuromodulation device, and the programming device can be an external programming device configured to be communicatively coupled to the implantable neuromodulation device via telemetry, as further discussed with reference to FIGS. 5 and 6. In another example, the neuromodulation device 212 can be an external neuromodulation device such as a Transcutaneous Electrical Neural Stimulation (TENS) device, the electrodes 211 can include surface electrodes such as skin patch electrodes, and the programming device can be an external programming device configured to be communicatively coupled to the implantable neuromodulation device via a wired or wireless link, or integrated with the external neuromodulation device. In still another example, the neuromodulation device 212 can be an external neuromodulation device, the electrodes 211 can include percutaneous electrodes, and the programming device can be an external programming device configured to be communicatively coupled to the implantable neuromodulation device via a wired or wireless link, or integrated with the external neuromodulation device. In various embodiments, an external neuromodulation device with surface and/or percutaneous electrodes can be used, for example, for delivering a test neuromodulation, delivering a therapeutic neuromodulation during a trial period, and delivering a short-term therapeutic neuromodulation.

In one embodiment, an external neuromodulation device with surface electrodes can be used during a trial period prior to a potential implantation of an implantable SCS system. A skin patch including the surface electrodes is placed over the patient's spine near the region where percutaneous electrodes will be placed for use during the trial period. The external neuromodulation device such as a dedicated External Trial Stimulator (ETC) and/or an external TENS device may be used to deliver stimulation energy to induce paresthesia and mapping dermatomal coverage of the pain sensation using one or more electrodes selected from the surface electrodes. The external neuromodulation device may also be programmed to deliver therapeutic neuromodulation (e.g., sub-threshold neuromodulation) through the percutaneous electrodes immediately following the placement of the percutaneous electrodes.

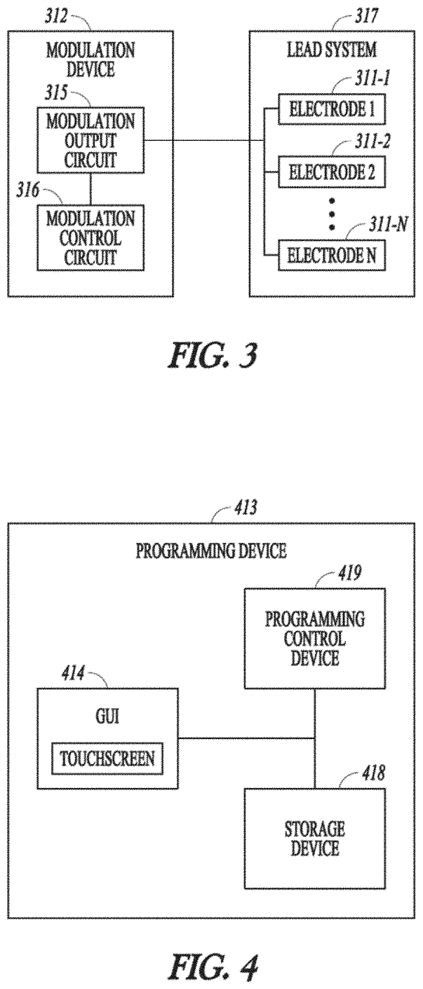

FIG. 3 illustrates an embodiment of a neuromodulation device 312, such as may be implemented in the neuromodulation system 210 of FIG. 2. The illustrated embodiment of the neuromodulation device 312 includes a neuromodulation output circuit 315 and a neuromodulation control circuit 316. Those of ordinary skill in the art will understand that the neuromodulation device 312 may include additional components such as sensing circuitry for patient monitoring and/or feedback control of the therapy, telemetry circuitry and power. The neuromodulation output circuit 315 produces and delivers neuromodulation pulses. The neuromodulation control circuit 316 controls the delivery of the neuromodulation pulses using the plurality of neuromodulation parameters. The combination of the neuromodulation output circuit 315 and neuromodulation control circuit 316 may collectively be referred to as a pulse generator. The lead system 317 includes one or more leads each configured to be electrically connected to neuromodulation device 312 and a plurality of electrodes 311-1 to 311-N (where N≥2) distributed in an electrode arrangement using the one or more leads. Each lead may have an electrode array consisting of two or more electrodes, which also may be referred to as contacts. Multiple leads may provide multiple electrode arrays to provide the electrode arrangement. Each electrode is a single electrically conductive contact providing for an electrical interface between neuromodulation output circuit 315 and tissue of the patient. The neuromodulation pulses are each delivered from the neuromodulation output circuit 315 through a set of electrodes selected from the electrodes 311-1 to 311-N. The number of leads and the number of electrodes on each lead may depend on, for example, the distribution of target(s) of the neuromodulation and the need for controlling the distribution of electric field at each target. In one embodiment, by way of example and not limitation, the lead system includes two leads each having eight electrodes. Some embodiments may use a lead system that includes one or more leads of the same or different types such as percutaneous leads, linear paddles, multiple-column paddles, or directional leads, among others.

The neuromodulation system may be configured to modulate spinal target tissue or other neural tissue. The configuration of electrodes used to deliver electrical pulses to the targeted tissue constitutes an electrode configuration, with the electrodes capable of being selectively programmed to act as anodes (positive), cathodes (negative), or left off (zero). In other words, an electrode configuration represents the polarity being positive, negative, or zero. An electrical waveform may be controlled or varied for delivery using electrode configuration(s). The electrical waveforms may be analog or digital signals. In some embodiments, the electrical waveform includes pulses. The pulses may be delivered in a regular, repeating pattern, or may be delivered using complex patterns of pulses that appear to be irregular. Other parameters that may be controlled or varied include amplitude, pulse width, rate (or frequency), or waveform of the electrical pulses. Each electrode configuration, along with the electrical pulse parameters, can be referred to as a “neuromodulation parameter set.” Each set of neuromodulation parameters, including fractionalized current distribution to the electrodes (as percentage cathodic current, percentage anodic current, or off), may be stored and combined into a neuromodulation program that can then be used to modulate multiple regions within the patient.

The number of electrodes available combined with the ability to generate a variety of complex electrical pulses, presents a huge selection of neuromodulation parameter sets to the clinician or patient. For example, if the neuromodulation system to be programmed has sixteen electrodes, millions of neuromodulation parameter sets may be available for programming into the neuromodulation system. Furthermore, for example SCS systems may have thirty-two electrodes that exponentially increases the number of neuromodulation parameters sets available for programming. To facilitate such selection, the clinician generally programs the neuromodulation parameters sets through a computerized programming system to allow the optimum neuromodulation parameters to be determined based on patient feedback or other means and to subsequently program the desired neuromodulation parameter sets.

Patient sensory input such as related to paresthesia perception may be used to program SCS therapy, such as by selecting or determining an appropriate neuromodulation parameter set. The paresthesia induced by neuromodulation and perceived by the patient may be located in approximately the same places of the patient body where pain is sensed and thus the target site of treatment. Conventionally, when leads are implanted within the patient, an operating room (OR) mapping procedure may be performed to apply neuromodulation to test placement of the leads and/or electrodes, thereby assuring that the leads and/or electrodes are implanted in effective locations within the patient.

Once the leads are correctly positioned, a fitting procedure, which may be referred to as a navigation session, may be performed to program the external control device, and if applicable the neuromodulation device, with a set of neuromodulation parameters that best addresses the painful site. Thus, the navigation session may be used to pinpoint the volume of activation (VOA) or areas correlating to the pain. The procedure may be implemented to target the tissue during implantation, or after implantation should the leads gradually or unexpectedly move that would otherwise relocate the neuromodulation energy away from the target site. By reprogramming the neuromodulation device (typically by independently varying the neuromodulation energy on the electrodes), the VOA can often be moved back to the effective pain site without having to re-operate on the patient in order to reposition the lead and its electrode array. According to various embodiments discussed in this document, the dermatomal coverage may be quantified using pain targeting metrics of spatial correspondence between body sites of pain and body sites responsive to diagnostic stimulation. A user interface may allow a user (e.g., a clinician) to adjust a stimulation setting and see an effect of the pain-targeting outcome at each dermatomal compartment. This may not only improve precision of pain targeting and thus lead to a better therapeutic outcome, but may also save a system operator's time and ease the burden of searching for stimulation sweet spot and optimizing target neuromodulation field design.

FIG. 4 illustrates an embodiment of a programming device 413, such as may be implemented as the programming device 213 in the neuromodulation system of FIG. 2, The programming device 413 includes a storage device 418, a programming control circuit 419, and a GUI 414. The programming control circuit 419 generates the plurality of neuromodulation parameters that controls the delivery of the neuromodulation pulses according to the pattern of the neuromodulation pulses. In various embodiments, the GUI 414 includes any type of presentation device, such as interactive or non-interactive screens, and any type of user input devices that allow the user to program the neuromodulation parameters, such as touchscreen, keyboard, keypad, touchpad, trackball, joystick, and mouse. The storage device 418 may store, among other things, neuromodulation parameters to be programmed into the neuromodulation device. The programming device 413 may transmit the plurality of neuromodulation parameters to the neuromodulation device. In some embodiments, the programming device 413 may transmit power to the neuromodulation device. The programming control circuit 419 may generate the plurality of neuromodulation parameters. In various embodiments, the programming control circuit 419 may check values of the plurality of neuromodulation parameters against safety rules to limit these values within constraints of the safety rules.

In various embodiments, circuits of neuromodulation, including its various embodiments discussed in this document, may be implemented using a combination of hardware, software and firmware. For example, the circuit of a GUI, neuromodulation control circuit, and programming control circuit, including their various embodiments discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

FIG. 5 illustrates, by way of example, an implantable neuromodulation system and portions of an environment in which system may be used. The system is illustrated for implantation near the spinal cord. However, neuromodulation system may be configured to modulate other neural targets such as may be useful for delivering other therapies. The system 520 includes an implantable system 521, an external system 522, and a telemetry link 523 providing for wireless communication between implantable system 521 and external system 522. The implantable system is illustrated as being implanted in the patient's body. The implantable system 521 includes an implantable neuromodulation device (also referred to as an implantable pulse generator, or IPG) 512, a lead system 517, and electrodes 511. The lead system 517 includes one or more leads each configured to be electrically connected to the neuromodulation device 512 and a plurality of electrodes 511 distributed in the one or more leads. In various embodiments, the external system 402 includes one or more external (non-implantable) devices each allowing a user (e.g. a clinician or other caregiver and/or the patient) to communicate with the implantable system 521. In some embodiments, the external system 522 includes a programming device intended for a clinician or other caregiver to initialize and adjust settings for the implantable system 521 and a remote control device intended for use by the patient. For example, the remote control device may allow the patient to turn a therapy on and off and/or adjust certain patient-programmable parameters of the plurality of neuromodulation parameters.

The neuromodulation lead(s) of the lead system 517 may be placed adjacent, i.e., resting near, or upon the dura, adjacent to the spinal cord area to be stimulated. For example, the neuromodulation lead(s) may be implanted along a longitudinal axis of the spinal cord of the patient. Due to the lack of space near the location where the neuromodulation lead(s) exit the spinal column, the implantable neuromodulation device 512 may be implanted in a surgically made pocket either in the abdomen or above the buttocks, or may be implanted in other locations of the patient's body. The lead extension(s) may be used to facilitate the implantation of the implantable neuromodulation device 512 away from the exit point of the neuromodulation lead(s).

FIG. 6 illustrates, by way of example, an embodiment of a SCS system, which also may be referred to as a Spinal Cord Neuromodulation (SCM) system. The SCS system 624 may generally include a plurality (illustrated as two) of implantable neuromodulation leads 625, an implantable pulse generator (IPG) 626, an external remote controller RC 627, a clinician's programmer (CP) 628, and an external trial modulator (ETM) 629. The IPG 626 may be physically connected via one or more percutaneous lead extensions 630 to the neuromodulation leads 625, which carry a plurality of electrodes 631. As illustrated, the neuromodulation leads 625 may be percutaneous leads with the electrodes arranged in-line along the neuromodulation leads. Any suitable number of neuromodulation leads can be provided, including only one, as long as the number of electrodes is greater than two (including the IPG case function as a case electrode) to allow for lateral steering of the current. Alternatively, a surgical paddle lead can be used in place of one or more of the percutaneous leads. The IPG 626 includes pulse generation circuitry, also referred to as a pulse generator, that delivers electrical neuromodulation energy in the form of a pulsed electrical waveform (i.e., a temporal series of electrical pulses) to the electrodes in accordance with a set of neuromodulation parameters.

The ETM 629 may also be physically connected via the percutaneous lead extensions 632 and external cable 633 to the neuromodulation leads 625. The ETM 629 may have similar pulse generation circuitry as the IPG 626 to deliver electrical neuromodulation energy to the electrodes accordance with a set of neuromodulation parameters. The ETM 629 is a non-implantable device that is used on a trial basis after the neuromodulation leads 625 have been implanted and prior to implantation of the IPG 626, to test the responsiveness of the neuromodulation that is to be provided. Functions described herein with respect to the IPG 626 can likewise be performed with respect to the ETM 629.

The RC 627 may be used to telemetrically control the ETM 629 via a bi-directional RF communications link 634, The RC 627 may be used to telemetrically control the IPG 626 via a bi-directional RF communications link 635. Such control allows the IPG 626 to be turned on or off and to be programmed with different neuromodulation parameter sets. The IPG 626 may also be operated to modify the programmed neuromodulation parameters to actively control the characteristics of the electrical neuromodulation energy output by the IPG 626. A clinician may use the CP 628 to program neuromodulation parameters into the IPG 626 and ETM 629 in the operating room and in follow-up sessions.

The CP 628 may indirectly communicate with the IPG 626 or ETM 629, through the RC 627, via an IR communications link 636 or other link. The CP 628 may directly communicate with the IPG 626 or ETM 629 via an RF communications link or other link (not shown). The clinician detailed neuromodulation parameters provided by the CP 628 may also be used to program the RC 627, so that the neuromodulation parameters can be subsequently modified by operation of the RC 627 in a stand-alone mode (i.e., without the assistance of the CP 628). Various devices may function as the CP 628. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 628. Alternatively, such programming methodologies can be performed using firmware or hardware. In any event, the CP 628 may actively control the characteristics of the electrical neuromodulation generated by the IPG 626 to allow the desired parameters to be determined based on patient feedback or other feedback and for subsequently programming the IPG 626 with the desired neuromodulation parameters. To allow the user to perform these functions, the CP 628 may include a user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g. CP) may be programmed to provide display screen(s) that allow the clinician to, among other functions, to select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical neuromodulation energy output by the neuromodulation leads, and select and program the IPG with neuromodulation parameters in a surgical or clinical setting.

An external charger 637 may be a portable device used to transcutaneously Charge the IPG via a wireless link such as an inductive link 638. Once the IPG has been programmed, and its power source has been charged by the external charger or otherwise replenished, the IPG may function as programmed without the RC or CP being present.

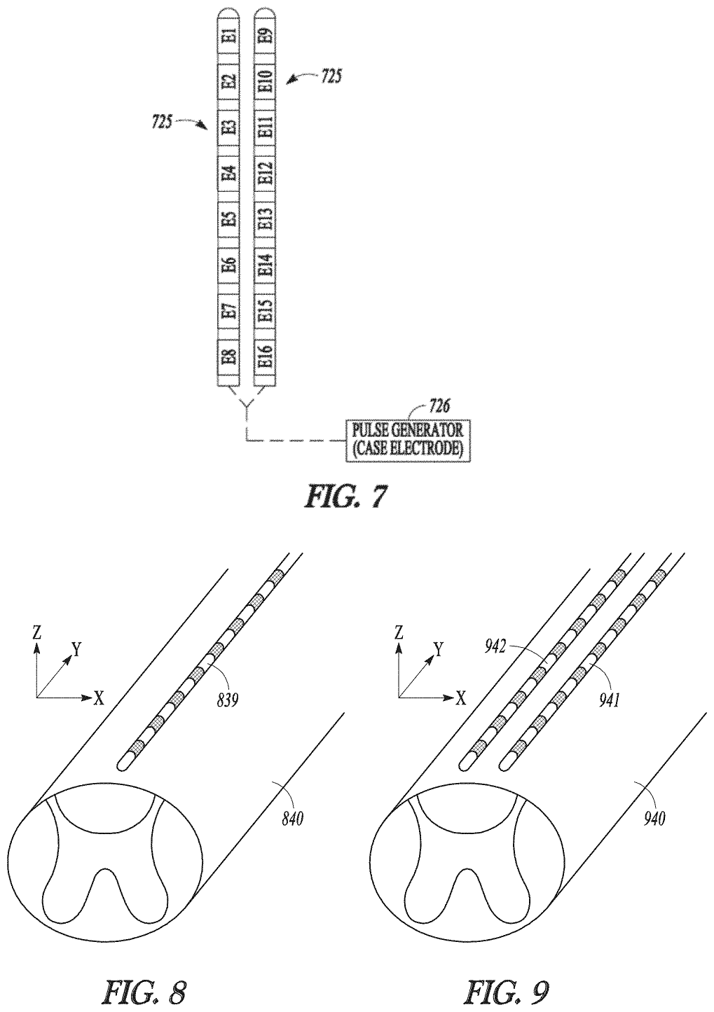

FIG. 7 illustrates, by way of example, some features of the neuromodulation leads 725 and a pulse generator 726. The pulse generator 726 may be an implantable device (IPG) or may be an external device such as may be used to test the electrodes during an implantation procedure. In the illustrated example, one of the neuromodulation leads has eight electrodes (labeled E1-E8), and the other neuromodulation lead has eight electrodes (labeled E9-E16). The actual number and shape of leads and electrodes may vary for the intended application. An implantable pulse generator (IPG) may include an outer case for housing the electronic and other components. The outer case may be composed of an electrically conductive, biocompatible material, such as titanium, that forms a hermetically-sealed compartment wherein the internal electronics are protected from the body tissue and fluids. In some cases, the outer case may serve as an electrode (e.g. case electrode). The IPG may include electronic components, such as a controller/processor (e.g., a microcontroller), memory, a battery, telemetry circuitry, monitoring circuitry, neuromodulation output circuitry, and other suitable components known to those skilled in the art. The microcontroller executes a suitable program stored in memory, for directing and controlling the neuromodulation performed by IPG.

Electrical neuromodulation energy is provided to the electrodes in accordance with a set of neuromodulation parameters programmed into the pulse generator. The electrical neuromodulation energy may be in the form of a pulsed electrical waveform. Such neuromodulation parameters may comprise electrode combinations, which define the electrodes that are activated as anodes (positive), cathodes (negative), and turned off (zero), percentage of neuromodulation energy assigned to each electrode (fractionalized electrode configurations), and electrical pulse parameters, which define the pulse amplitude (measured in milliamps or volts depending on whether the pulse generator supplies constant current or constant voltage to the electrode array), pulse width (measured in microseconds), pulse rate (measured in pulses per second), and burst rate (measured as the neuromodulation on duration X and neuromodulation off duration Y). The electrical pulse parameters may define an intermittent neuromodulation with “ON” periods of time where a train of two or more pulses are delivered and “OFF” periods of time where pulses are not delivered. Electrodes that are selected to transmit or receive electrical energy are referred to herein as “activated,” while electrodes that are not selected to transmit or receive electrical energy are referred to herein as “non-activated.”

Electrical neuromodulation occurs between or among a plurality of activated electrodes, one of which may be the IPG case. The system may be capable of transmitting neuromodulation energy to the tissue in a monopolar or multipolar (e.g., bipolar, tripolar, etc.) fashion. Monopolar neuromodulation occurs when a selected one of the lead electrodes is activated along with the case of the IPG, so that neuromodulation energy is transmitted between the selected electrode and case. Any of the electrodes E1-E16 and the case electrode may be assigned to up to k possible groups or timing “channels.” In one embodiment, k may equal four. The timing channel identifies which electrodes are selected to synchronously source or sink current to create an electric field in the tissue to be stimulated. Amplitudes and polarities of electrodes on a channel may vary. In particular, the electrodes can be selected to be positive (anode, sourcing current), negative (cathode, sinking current), or off (no current) polarity in any of the k timing channels. The IPG may be operated in a mode to deliver electrical neuromodulation energy that is therapeutically effective and causes the patient to perceive delivery of the energy (e.g. therapeutically effective to relieve pain with perceived paresthesia), and may be operated in a sub-perception mode to deliver electrical neuromodulation energy that is therapeutically effective and does not cause the patient to perceive delivery of the energy (e.g. therapeutically effective to relieve pain without perceived paresthesia).

The IPG may be configured to individually control the electrical current flowing through each of the electrodes. For example, a current generator may be configured to selectively generate individual current-regulated amplitudes from independent current sources for each electrode. In some embodiments, the pulse generator may have voltage regulated outputs. While individually programmable electrode amplitudes are desirable to achieve fine control, a single output source switched across electrodes may also be used, although with less fine control in programming. Neuromodulators may be designed with mixed current and voltage regulated devices.

FIGS. 8-9 are schematic views of embodiments of neuromodulation lead placement on a patient's spinal cord. Specifically, FIG. 8 is a schematic view of a single electrical neuromodulation lead 839 implanted over approximately the longitudinal midline of the spinal cord 840. It is understood that additional leads or lead paddle(s) may be used, such as may be used to provide a wider electrode arrangement and/or to provide the electrodes closer to dorsal horn elements, and that these electrode arrays also may implement fractionalized current. FIG. 9 illustrates an embodiment where two electrical neuromodulation leads are implanted near the spinal cord. A first electrical neuromodulation lead 941 is implanted more laterally with respect to a spinal cord 940, thereby placing it proximate the dorsal horn of the spinal cord 940. A second electrical neuromodulation lead 942 is implanted more medially with respect to the spinal cord, thereby placing it proximate the dorsal column of the spinal cord 940.

Placement of the lead more proximal to the DH than the DC may be desirable to preferentially stimulate DH elements over DC neural elements for a sub-perception therapy. Lead placement may also enable preferential neuromodulation of dorsal roots over other neural elements. Any other plurality of leads or a multiple column paddle lead can also be used. Longitudinal component of the electrical field is directed along the y-axis depicted in each of FIGS. 8-9 and a transverse component of the electrical field is directed along the x-axis depicted in each of FIGS. 8-9.

It is to be understood that additional neuromodulation leads or paddle(s) of the same or different types may be used, such as may be used to provide a wider electrode arrangement and/or to provide the electrodes closer to dorsal horn elements. Some embodiments may include directional leads with one or more directional electrodes. A directional electrode may extend less than 360 degrees about the circumference of a lead body. For example, a row of two or more directional electrodes (e.g. “segmented electrodes”) may be positioned along the circumference of the lead body. Activating select ones of the segmented electrodes may help extend and shape the field in a preferred direction. In some examples, the neuromodulation leads or paddles maybe placed at regions more caudal to the end of the spinal cord, and the electrode arrays on the neuromodulation lead also may implement fractionalized current.

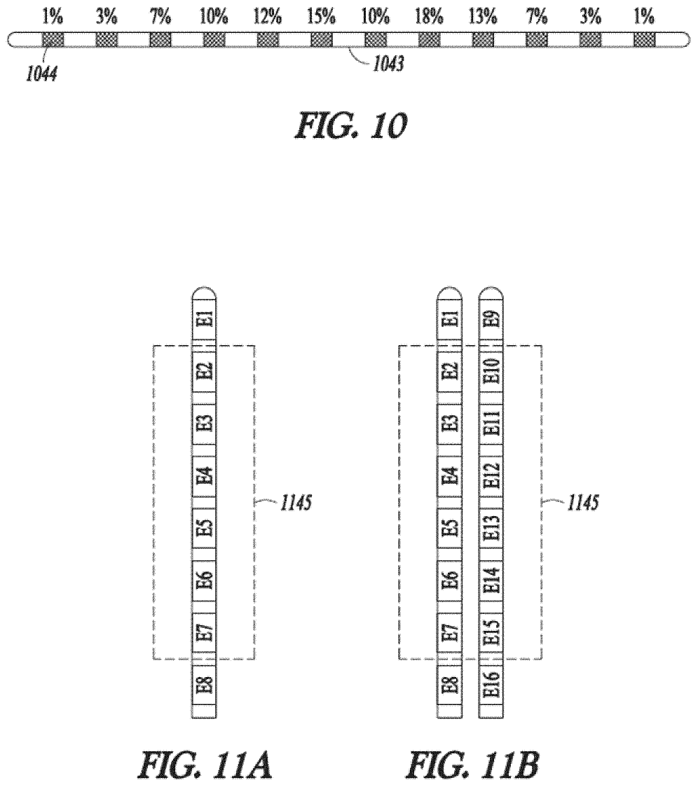

FIG. 10 is a schematic view of an electrical neuromodulation lead 1043 showing an example of the fractionalization of the anodic current delivered to the electrodes on the electrical neuromodulation lead. These figures illustrate fractionalization using monopolar neuromodulation where a case electrode of the IPG is the only cathode, and carries 100% of the cathodic current. The fractionalization of the anodic current shown in FIG. 10 does not deliver an equal amount of current to each electrode 1044, because this embodiment takes into account electrode/tissue coupling differences, which are the differences in how the tissue underlying each electrode reacts to electrical neuromodulation. Also, the ends of the portion of the electrical neuromodulation lead include electrodes having lower gradient in the longitudinal direction. The magnitude of the electrical field tapers down at the ends of the electrical neuromodulation lead. Fractionalization of the current may accommodate variation in the tissue underlying those electrodes. The fractionalization across the electrical neuromodulation lead can vary in any manner as long as the total of fractionalized currents equals 100%. Various embodiments described herein implement a programmed algorithm to determine the appropriate fractionalization to achieve a desired neuromodulation field property.

Neuromodulation thresholds vary from patient to patient and from electrode to electrode within a patient. An electrode/tissue coupling calibration of the electrodes may be performed to account for these different neuromodulation thresholds and provide a more accurate fractionalization of the current between electrodes. For example, perception threshold may be used to normalize the electrodes. The RC or the CP may be configured to prompt the patient to actuate a control element, once paresthesia is perceived by the patient. In response to this user input, the RC or the CP may be configured to respond to the user input by storing the neuromodulation signal strength when the control element is actuated. Other sensed parameter or patient-perceived neuromodulation values (e.g. constant paresthesia, or maximum tolerable paresthesia) may be used to provide the electrode/tissue coupling calibration of the electrodes.

The SCS system may be configured to deliver different electrical fields to achieve a temporal summation of neuromodulation. The electrical fields can be generated respectively on a pulse-by-pulse basis. For example, a first electrical field can be generated by the electrodes (using a first current fractionalization) during a first electrical pulse of the pulsed waveform, a second different electrical field can be generated by the electrodes (using a second different current fractionalization) during a second electrical pulse of the pulsed waveform, a third different electrical field can be generated by the electrodes (using a third different current fractionalization) during a third electrical pulse of the pulsed waveform, a fourth different electrical field can be generated by the electrodes (using a fourth different current fractionalized) during a fourth electrical pulse of the pulsed waveform, and so forth. These electrical fields may be rotated or cycled through multiple times under a timing scheme, where each field is implemented using a timing channel. The electrical fields may be generated at a continuous pulse rate, or may be bursted on and off. Furthermore, the interpulse interval (i.e., the time between adjacent pulses), pulse amplitude, and pulse duration during the electrical field cycles may be uniform or may vary within the electrical field cycle.

Some embodiments are configured to determine a neuromodulation parameter set to create a stimulation field definition to reduce or minimize neuromodulation of non-targeted tissue (e.g. DC tissue). The neuromodulation field may be shaped by using multiple independent current control (MICC) or multiple independent voltage control to guide the estimate of current fractionalization among multiple electrodes and estimate a total amplitude that provide a desired strength. For example, the neuromodulation field may be shaped to enhance the neuromodulation of DH neural tissue and to minimize the neuromodulation of DC tissue. A benefit of MICC is that MICC accounts for various in electrode-tissue coupling efficiency and perception threshold at each individual contact, so that “hot-spot” stimulation is eliminated.

FIGS. 11A-11B illustrate, by way of example and not limitation, electrode arrangements (e.g. E1-E8 in FIG. 11A and E1-E16 in FIG. 11B) and test regions 1145 of neural tissue along the electrode arrangements. These test regions 1145 may extend across the entire electrode arrangement. In some embodiments, the test regions may extend along only a portion of the electrode arrangement. By way of example, some embodiments may allow a user to select the test region and thus select the portion of the electrode arrangement to be tested. In the example illustrated in FIG. 11A the test region is neural tissue along the E2 to E7 electrodes, and in the example illustrated in FIG. 11B the test region is neural tissue along the E2 through E7 and the E10 to E15 electrodes.

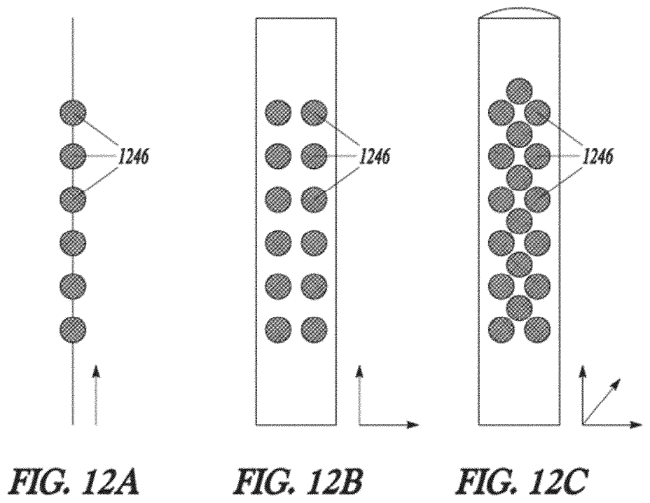

The current applied to the electrode arrangement may be fractionalized, using different neuromodulation parameter sets, to change the portion of the neural tissue that is modulated. Thus, there may be many neural tissue locations that can be targeted with the test region of neural tissue adjacent to the electrode arrangement. FIGS. 12A-12C illustrate, by way of example and not limitation, neural tissue locations 1246 that may be targeted within the test region in one, two and three dimensions, respectively. In the one-dimensional example illustrated in FIG. 12A, the neural locations that may be targeted may simply be a line of potential targets such as may be observed from a single lead with a linear arrangement of electrodes. In the two dimensional example illustrated in FIG. 12B the neural locations that may be targeted may be considered to lie in a plane proximate to the electrode arrangement. In the three-dimensional example illustrated in FIG. 12C, the neural locations that may be targeted may be considered to be a volume of tissue proximate to the electrode arrangement. By way of example, the two-dimensional and three-dimensional test regions may be implemented using two or more leads of electrodes. Thus, the test regions may be relatively simple or complex shapes, and may include relatively few or relatively many locations to be tested.

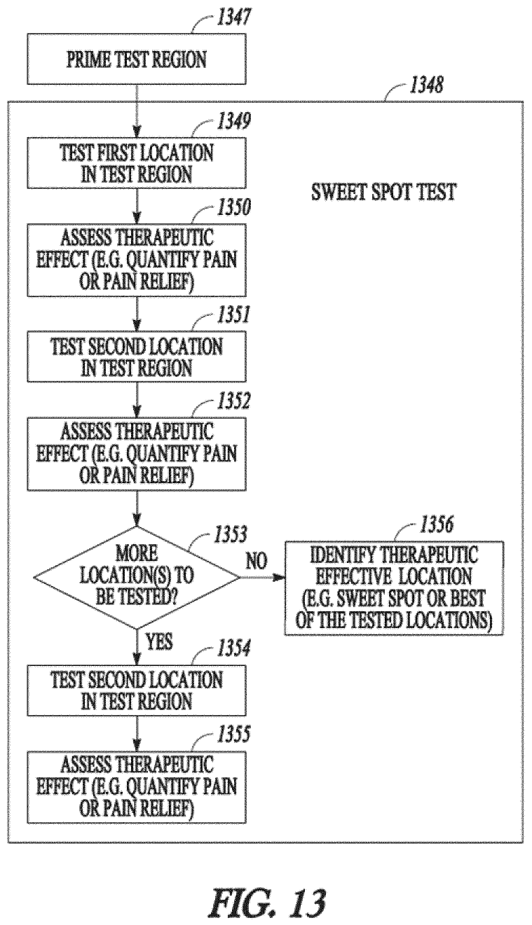

FIG. 13 illustrates an example of a method 1300 for finding a sweet spot for sub-perception neuromodulation. In the illustrated example, a test region is primed with the sub-perception neuromodulation field 1347, and the sweet-spot test is performed 1348 to find location of neural tissue that is therapeutically effective when targeted with sub-perception neuromodulation. The sweet spot test may involve a manual process to reprogram the neuromodulation field parameter set with different values to change the targeted location of the neuromodulation field. In some embodiments of the test, the targeted location is automatically changed (e.g. trolled) by automatically changing values of the neuromodulation field parameter set. Some embodiments may semi-automatically change values of the neuromodulation field parameter set to change the targeted location of the neuromodulation field.

At 1349, a first location in the test region is tested by focusing the neuromodulation field onto the first location. At 1350, the therapeutic effect of modulating the first location is assessed. In an example where the therapy is a therapy to alleviate pain, the patient may provide this assessment by quantifying a level of pain or level of pain relief that they are experiencing. In some examples, a biomarker is used to provide an assessment of the therapeutic efficacy of the neuromodulation field focused on the tested location. At 1351, the neuromodulation field parameter set is changed to change the focus of the neuromodulation field to test a second location in the test region. At 1352, the therapeutic effect of modulating the second location is assessed. If more location(s) are to be tested, as illustrated at 1353, the process may continue to 1354 to test the next location. The process can then continue at 1355 to assess the therapeutic effect of the next location. The process may determine or identify the location(s) that are therapeutically effective 1356 by evaluating the quantified effects of the therapy. In some embodiments, the quantified effects may be compared to each other to identify the tested location that has the best therapeutic effect or one of the best therapeutic effects.

The method 1300 may be performed to test relatively small locations using a more narrowly focused neuromodulation field such as generally illustrated above in FIGS. 12A-12C, or may be used to test relatively larger locations of neural tissue using a more uniform (less focused) neuromodulation field. The test of larger locations may be followed by a more focused test or tests within one of the larger location. In an example, a test may begin with full-lead then use a search algorithm to reduce the span and improve energy efficiency. This can be done from the RC or CP, or in the IPG with RC feedback. The proposed algorithms may rely on some form of feedback indicating the effectiveness of the neuromodulation. For example, a patient may provide feedback regarding pain relief. Feedback may also provide a biomarker signal. In another example, the test may include a step of confirming that the neuromodulation along the full lead is effective, then focusing the neuromodulation along a portion of the lead. Thus, for example, a generally uniform neuromodulation field may be provided along this smaller portion of the lead. This field is still broad as it may be provided across an area with multiple electrode contacts, but it is less than the entire electrode arrangement using electrode array(s) on the lead(s).

FIG. 14 illustrates, by way of example, aspects of a binary search routine as a rostrocaudal focus routine. A first subset of electrodes that define a first partial lead search region can be tested to determine if the neuromodulation is effective using the first subset 1457. If it is effective, the first subset of electrodes that define the first partial lead search region may be used to deliver the neuromodulation 1458 or for further more focused tests. If it is not effective, then a second subset of electrodes that define a second partial lead search region may be tested to determine if the second subset of electrodes is effective 1459. If it is effective, the second subset of electrodes that define the second partial lead search region may be used to deliver the neuromodulation 1458. If it is not effective, then a third (or nth) subset of electrodes that define a third (or nth) partial lead search region may be tested to determine if the third (or nth) subset of electrodes is effective 1460. If it is effective, the third (or nth) subset of electrodes that define the third (or nth) partial lead search region may be used to deliver the neuromodulation 1458. If it is not effective, then the binary search process may return to the full list of electrodes 1461, which was previously determined to be effective. At least some of the subsets of electrodes may be exclusive of each other. At least some of the subsets of electrodes may intersect with each other. In some embodiments, at least two subsets are exclusive, and at least one subset has an intersection with another subset.

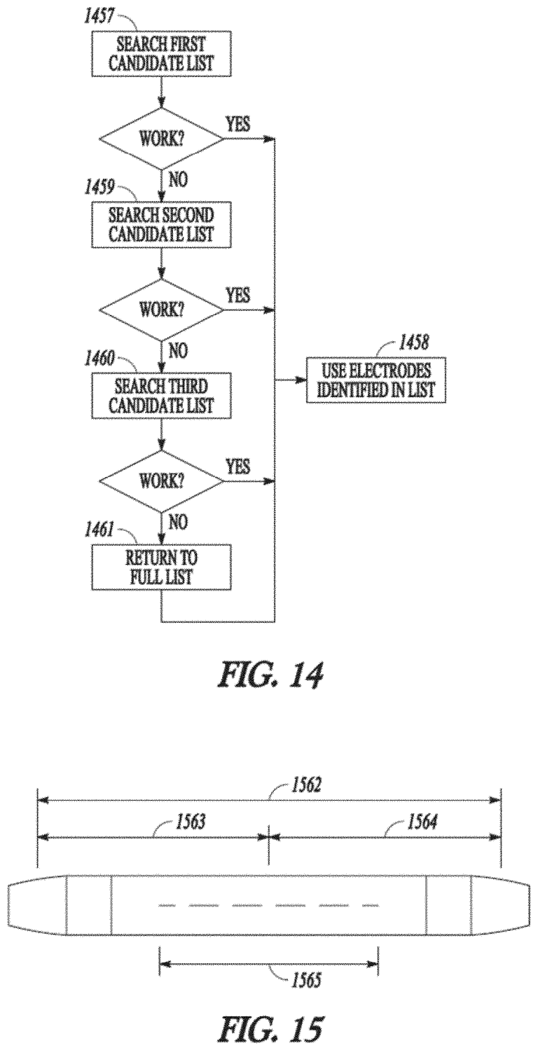

FIG. 15 illustrates an example of the binary search routine. The lead has a full span 1562 which may be split into three partial lead search regions 1563, 1564 and 1565, each partial search region including a corresponding subset of electrodes. By way of example and not limitation, the first and second subsets 1563 and 1564 of electrodes may be mutually exclusive, and third subset 1565 may include an intersection with the first subset and also may include an intersection with the second set. In an example, the full lead may be bifurcated to provide the first partial lead search region 1563 on a first side of the lead (e.g. left end of electrode array to middle) and the second partial lead search region 1564 on a second side of the lead (e.g. right end of the electrode array to middle). The third partial lead search region 1565 may partially overlap each of the first and second partial lead search regions. Thus, the partial lead search regions may define a first end region, a second end region and a middle region of the lead.

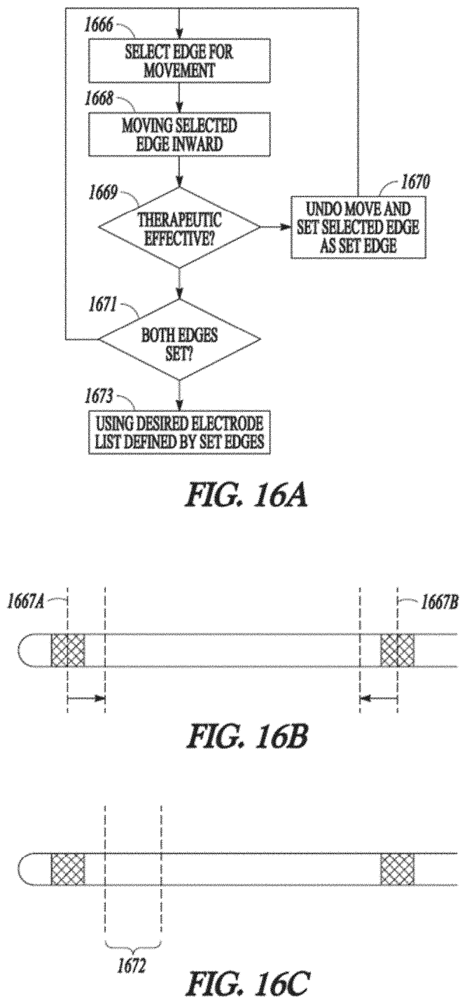

FIGS. 16A-16C illustrate, by way of example, an edge search routine. The edge search routine progressively moves each edge of the active electrodes in the array, toward the middle and confirms that the neuromodulation remains effective with the moves. Thus, a first edge can be moved toward the center until the next move toward the center causes the neuromodulation to be ineffective; and a second edge can be moved toward the center until the next move toward the center causes the neuromodulation to be ineffective.

For example, the edge search routine may include selecting an edge of the electrode arrangement (e.g. array) for movement 1666. The selected edge may be one of the two edges 1667A or 1667B illustrated in FIG. 16B. However, there can be more than two edges if more than two regions are being focused. The selected edge is moved inward 1668 toward the other edge for the region of interest. If the reduced set of electrodes is no longer therapeutically effective 1669, then the previous move can be undone and that edge can be set so that is no longer is capable of being selected for movement 1670. The process can return to 1666 to attempt to move the other edge(s). If the reduced set of electrodes continues to be therapeutically effective 1669, then the process returns to 1666 to continue moving edges until such time as all of the edges are set 1671. The final reduced set 1672 of electrodes can be used 1673 to deliver the neuromodulation energy.

According to various embodiments, the programmed system may be configured with a neuromodulation focus routine such as a rostrocaudal focus routine to allow a user to select the desired electrodes for the neuromodulation to be more specific to the desired physiological area. Some embodiments may allow non-contiguous spans to be selected as a result of initial programming and/or neuromodulation refinement later on.

The neuromodulation field may be moved from location to location using an automatic trolling process or through patient control. Candidate trolling algorithms include a monopolar troll (e.g., anodic trolling or cathodic trolling) or a bipolar troll or a multipolar troll. The troll can be done with MICC or multiple independent voltage control, or with a timing channel interleaving technique. MICC enables the locus of the neuromodulation to be gradually moved across along the lead or within the array of electrodes. The interleaving of timing channels allows different electrode(s) in different timing channels. Values of stimulation parameter(s) (e.g. amplitude) in the timing channels can be adjusted. Thus by way of example and not limitation, if a monopolar neuromodulation is delivered using a first electrode in a first channel and another monopolar neuromodulation is delivered using a second electrode adjacent to the first electrode in a second channel, then the amplitude of the monopolar neuromodulation in the first channel may be incrementally reduced as the amplitude of the monopolar neuromodulation may be increase in the second channel. In this matter, the locus of the neuromodulation may be gradually adjusted.

Various embodiments troll a neuromodulation field, using an arrangement of electrodes on at least one lead, through neural tissue positions, and perform a quantification procedure multiple times as the neuromodulation field is trolled through the positions. The quantification procedure identifies when the neuromodulation field provides a therapeutic effect (e.g. pain relief). The quantification procedure may include receiving a marking signal that indicates that a neuromodulation intensity achieved the therapeutic effect, and storing a value for the therapeutic effect as well as neuromodulation field parameter data. The neuromodulation intensity may include neuromodulation parameters that affect the patient's perception of the neuromodulation energy. These parameters may include pulse width, rate, amplitude, distribution of current, and electrode polarity (cathode v. anode). By way of example and not limitation, the storage of the parameter data may be in a temporary storage such as but not limited to cache or RAM or in permanent/persistent storage such as but not limited to ROM, a memory device such a hard drive, optical disc, thumb drive, or cloud storage. The quantification process may include receiving a titration signal that indicates an instruction to adjust neuromodulation intensity, and adjusting the neuromodulation intensity in response to receiving the titration signal. The titration signal may be initiated by a patient, or by a clinician or other user who is responding to patient responses.

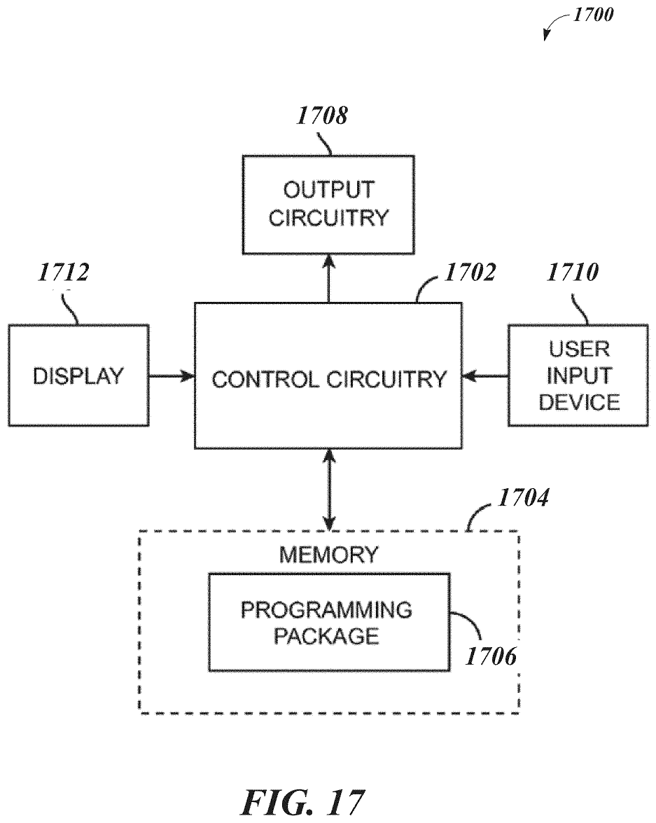

FIG. 17 is a diagram 1700 illustrating portions of a clinician's programmer which can be an embodiment of the CP 628 (FIG. 6), The CP 1700 includes a control circuitry 1702 (e.g., a central processor unit (CPU)) and memory 1704 that stores a stimulation programming package 1706, which can be executed by the control circuitry 1702 to allow the user to program the IPG 626 (FIG. 6), and RC 627 (FIG. 6). The CP 1700 further includes output circuitry 1708 (e.g., via the telemetry circuitry of the RC 627) for downloading stimulation parameters to the IPG 626 and RC 627 and for uploading stimulation parameters already stored in the memory of the RC 627, via the telemetry circuitry of the RC 627.

Execution of the programming package 1706 by the control circuitry 1702 provides a multitude of display screens shown on display 1712 that can be navigated through via use of user input device 1710. These display screens allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical stimulation energy output by the leads, and select and program the IPG 626 with stimulation parameters in both a surgical setting and a clinical setting.

In various embodiments, execution of the programming package 1706 provides a user interface that conveniently allows a user to program the IPG 626 to produce a user-customized stimulation field, which may include the placement and movement of customized target poles. In various examples, the programming package 1706, when executed by control circuitry 1702, implements a set of engines for facilitating the user interface in which fields or target poles may be defined, mapping the field or target pole definitions to physical electrodes and electrical energy application parameters for establishing the defined fields and target poles, supervising the establishment and variation of the fields and target poles to comply with safety and other defined constraints, and optimizing the energy utilization in the operation of the IPG 626.

In the examples described above, and in various other embodiments, the components described herein are implemented as engines, circuits, components, modules, or other structures, which for the sake of consistency are termed engines, although it will be understood that these terms may be used interchangeably. Engines may be hardware, software, or firmware communicatively coupled to one or more processors in order to carry out the operations described herein. Engines may be hardware engines, and as such, engines may be considered tangible entities capable of performing specified operations and may be configured or arranged in a certain manner. In an example, circuits may be arranged (e.g., internally or with respect to external entities such as other circuits) in a specified manner as an engine. In an example, the whole or part of one or more hardware processors may be configured by firmware or software (e.g., instructions, an application portion, or an application) as an engine that operates to perform specified operations. In an example, the software may reside on a machine-readable medium. In an example, the software, when executed by the underlying hardware of the engine, causes the hardware to perform the specified operations. Accordingly, the term hardware engine is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein.

Considering examples in which engines are temporarily configured, each of the engines need not be instantiated at any one moment in time. For example, where the engines comprise a general-purpose hardware processor core configured using software; the general-purpose hardware processor core may be configured as respective different engines at different times. Software may accordingly configure a hardware processor core, for example, to constitute a particular engine at one instance of time and to constitute a different engine at a different instance of time.

The present inventor has recognized an unmet need to optimally target the neuromodulation to the precise fibers that correspond to the source of pain to be treated or fibers mediating analgesia in the pain areas, while minimizing stimulation of other fibers in order to reduce or avoid side effects. However, neuromodulation with desirable precision can be challenging. For example, conventional dermatomal coverage of pain area, such as by mapping paresthesia sensation to the pain area, are not precise to individual dermatome compartments. Dermatomal coverage is generally based on qualitative characterization or quantification metrics with limited specificity to individual dermatomes. Optimizing a stimulation field definition and electrode configuration for delivering pain therapy with a high precision can be technically difficult and time-consuming.

One aspect of the embodiments discussed in this document is directed to optimizing a target neuromodulation field design, and applying a stimulation field to specific neural elements by an IPG such as neuromodulation device 212 (FIG. 2) or IPG 626 (FIG. 6). According to some embodiments, a neuromodulation system such as system 210 (FIG. 2) or the SCS system of FIG. 6, facilitates the creation of a target neuromodulation field by quantitative analysis of dermatomal coverage. An exemplary neuromodulation system includes data receiver configured to receive pain data including information of pain sites on a body of the patient, and to receive patient physiological data including information of body sites responsive to the applied first neuromodulation energy. The neuromodulation system includes a processor circuit configured to determine a pain distribution across a first set of dermatomal compartments using pain data (e.g., a patient pain drawing), and to determine a response distribution across a second set of dermatomal compartments using patient physiological data responsive to diagnostic stimulation (e.g., a paresthesia drawing or sensor data map). The neuromodulation system may generate a pain targeting metric (PTM) based on the pain distribution and the response distribution, and determine an optimal stimulation setting or a stimulation field definition for neuromodulation therapy using the PTM. In an example, in accordance with the optimal stimulation setting, the neuromodulation system may determine a stimulation field definition, which include one or more parameters such as field size, shape, and intensity, and field scaling and steering parameters. Neuromodulation energy (e.g., current) may be fractionalized among electrodes based on the stimulation field definition.

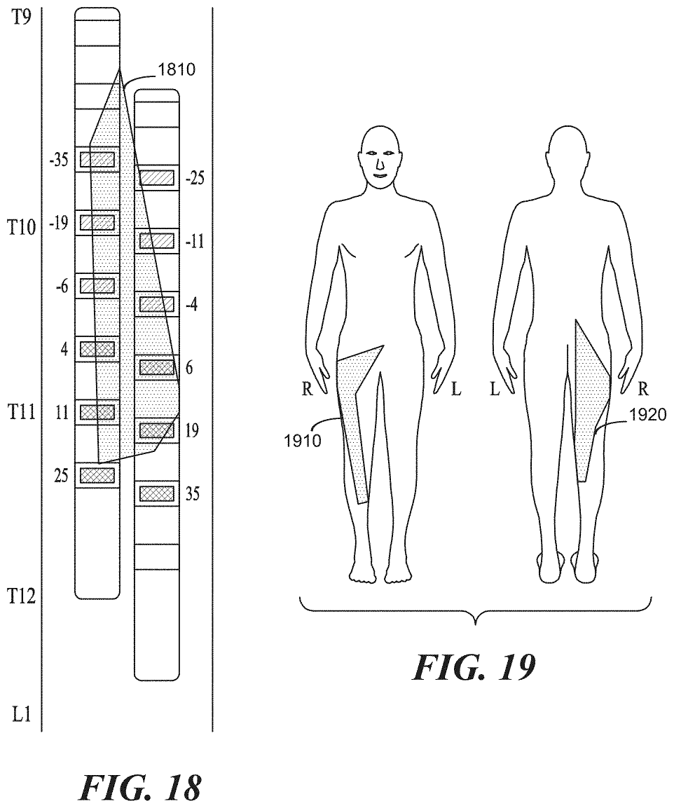

FIGS. 18 and 19 are diagrams illustrating, by way of example and not limitation, patient body sites where pain is felt (also referred to as pain sites), or body sites where the effect of neurostimulation delivery is felt (also referred to as response sites). The effect of neurostimulation delivery may be represented by patient sensory feedback (e.g., patient reporting of paresthesia perception), or by physiological data sensed by a sensor (e.g., electromyography, hereinafter “EMG”). The pain sites or the effected sites responsive to neurostimulation may each be drawn (e.g., free-hand drawing by the patient), derived from patient description (e.g., of pain sensation or paresthesia perception at different body sites) via a look up table or an algorithm, derived from a sensor data map (e.g., sensor signal amplitude or spectral intensity at different sensing sites, or signal spatio-temporal patterns between a set of sensors) via a look up table or an algorithm, or using a combination of the methods thereof. FIG. 18 illustrates an example of a drawing 1800 of target sites over a representation of a plurality of electrodes implanted in a patient. The target sites may be pain sites (in which case the drawing 1800 is referred to as a pain drawing), paresthesia sites (in which case the drawing 1800 is referred to a paresthesia drawing), or sensor response sites (in which case the drawing 1800 is referred to as a sensor data map). FIG. 19 illustrates, by way of example and not limitation, drawings representing target sites on an illustrative diagram of a human body including one or both of ventral or dorsal depiction of the body. The target sites are marked in a first drawing 1910 on a ventral depiction of the body, and in a second drawing 1920 on a dorsal depiction of the body. Similar to FIG. 18, the target sites can be pain sites, paresthesia sites, or sensor response sites. The first and second drawings 1910 and 1920 may each be a pain drawing, a paresthesia drawing, or a sensor data map. The drawings 1910 and 1920 may be provided to a neuromodulation system to generate an indication of spatial correspondence between the pain sites and the paresthesia sites, or an indication of spatial correspondence between the pain sites and the sensor response sites. The paresthesia sites or the sensor response sites may be mapped to one or more dermatome compartments on the skin. The mapping may be a point-by-point mapping via a look-up table or a dictionary/key system. The center, length, and width of the target linear field may be determined based on the spatial extent of the spinal cord region corresponding to the patient's reported pain region. A clinician or other specialist, a patient or a combination of persons may work together to highlight region of spinal cord and/or body where they want stimulation to be targeted (i.e. focus stimulation on anatomical correlate and/or reported site of pain). Internal look-up table and/or inverse algorithm with field “primitives” tied to specific regions/region sizes may be used to display and configure electrode settings according to this anatomically-based specification. The neuromodulation system may determine an optimal stimulation setting or a stimulation field definition based on the spatial correspondence indication.

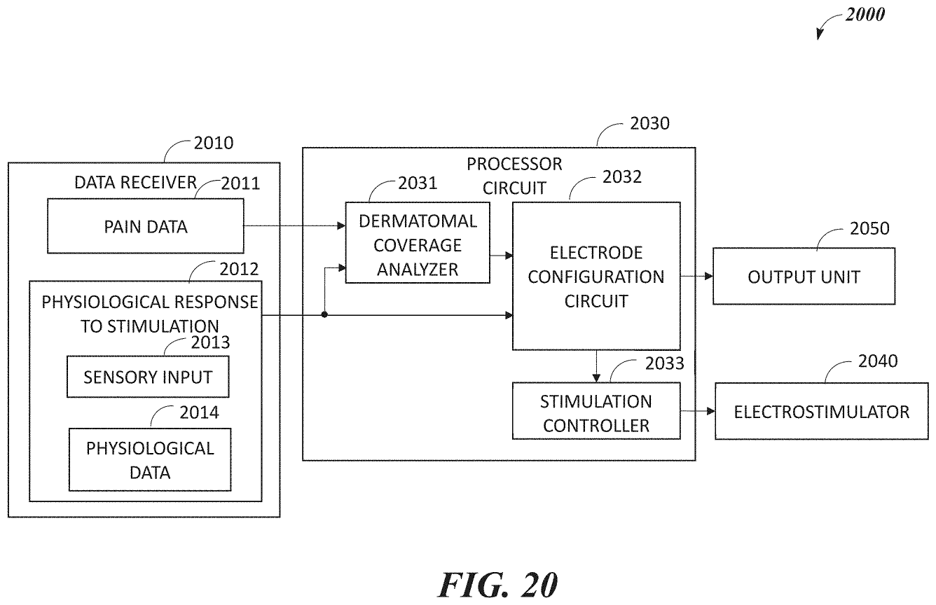

FIG. 20 is a block diagram illustrating, by way of example and not limitation, a neuromodulation system 2000 configured to apply electrostimulation to a target neural element, such as a spinal cord or an affiliated neural structure (e.g., spinal nerves, or ventral or dorsal roots), for pain control. The neuromodulation system 2000, which is an embodiment of the neuromodulation system 210, can include one or more of a data receiver 2010, a processor circuit 2030, an electrostimulator 2040, and an output unit 2050. Portions of the neuromodulation system 2000 may be implemented in the implantable system 521 or the external system 522.

The data receiver 2010 may receive, among other things, pain data 2011 and patient physiological response to stimulation 2012. The pain data 2011 may include information of anatomical locations of the pain felt by the patient (pain sites). The pain data 2011 may additionally include intensity of pain at various pain sites, quality of pain, or temporal pattern such as persistence of the pain at various pain sites, among other pain information. The pain data 2011 may be represented by texts, graphs, verbal description, among other means of representation. In an example, the pain data 2011 includes a pain drawing, such as the example illustrated in FIG. 19. The pain drawing may include pain markings that identify the locations where pain radiates or expands. The pain markings may additionally include symbols or numerals to characterize pain sensations, such as pain qualities (e.g., aching, numbness, burning, stabbing, or needle pain) or pain scores to distinguish pain intensities. In an example, the pain qualities may be represented by 0 for throbbing, 1 for stabbing, 2 for burning, 3 for pins and needles, 4 for crushing, and 5 for others. In an example, the pain scores may be represented by 0 for no pain, 1 for slight pain, 2 for moderate pain, 3 for severe pain, and 4 for extreme pain, etc. In addition to or in lieu of pain drawings, the pain data 2011 may include a patient questionnaire or pain verbal or written pain report, which may be translated manually or automatically into pain locations, pain qualities, and/or pain scores as discussed above. In some examples, the pain data 2011 may include reference data, such as a dermatome map that identifies (e.g., using labels, markers, or annotations) various skin areas corresponding to spinal nerves that innervates said skin areas.

The patient physiological response 2012 represents patient response to a first neuromodulation energy applied to a target neural element. The first neuromodulation energy can include electrostimulation, generated and delivered according to a programmed parameter setting, that induces patient sensory feedback such as paresthesia, or that elicits physiological responses that can be detected by a sensor. The first neuromodulation energy is used for targeting the pain area and determining dermatomal coverage of pain, and is referred to as diagnostic stimulation in this document. This is to distinguish from therapeutic stimulation that uses a different second neuromodulation energy delivered to the target neural element to relieve pain. In an example, the first neuromodulation energy is supra-perception stimulation energy, and the second neuromodulation energy is sub-perception stimulation energy. The first neuromodulation energy may induce paresthesia, evoke physiological response (e.g., sensor response) without paresthesia, or paresthesia with the evoked physiological response.

The first neuromodulation energy may be generated and delivered to the target neural element by the electrostimulator 2040, according to a stimulation setting via a set of electrodes at respective electrode locations. Examples of the target neural element may include a portion of a spinal cord, one or more spinal nerves, dorsal roots, or dorsal root ganglia. The stimulation setting may include a location of central point of stimulation (CPS) that represents a focal point of a stimulation field. The stimulation setting may additionally or alternatively include one or more stimulation parameters. Examples of the stimulation parameters may include a current amplitude or a voltage amplitude, a pulse width, a pulse waveform, a pulse rate, or a duty cycle, among other parameters.

The CPS location may be adjusted manually or automatically using a trolling process, such as using MICC or multiple independent voltage control, or with a timing channel interleaving technique. The CPS may be trolled up and down rostrocaudally) along the lead or paddle, and/or medio-laterally across the lead or paddle. This would move the locus of the neuromodulation across different spinal cord locations or different neural structures (e.g., spinal nerves, dorsal roots, or dorsal root ganglia). Trolling may be performed using a monopolar troll (e.g., anodic trolling or cathodic trolling), a bipolar troll, or a multipolar troll algorithm. In an example, trolling may be activated and manipulated manually by a system operator (e.g., a clinician) through a programmer device, such as one of the programming device 213, 413, or 628. Alternatively, trolling may be performed automatically, such as by moving the locus of the neuromodulation in a specific direction and delivering electrostimulation at specific locations within a specified permissible extent (e.g., a lumbar region).

The electrostimulator 2040 can stimulate the target tissue at each of a plurality of manually or automatically set CPS locations to induce paresthesia or to elicit a physiological response. At a CPS location, one or more stimulation parameters (e.g., pulse rate, amplitude, stimulation rate, etc.) may be adjusted, and neuromodulation energy may be delivered in accordance with the adjusted stimulation parameters. In an example, different values may be manually programmed by a user, or automatically selected from pre-determined parameter values such as by the stimulation controller 2033. In an example, the stimulation controller 2033 may trigger the electrostimulator 2040 to deliver the first neuromodulation energy according to a paresthesia induction protocol, such as scanning through a range of pre-determined values of a stimulation parameter (e.g., pulse widths of 100 μs, 300 μs and 500 μs), or a combination of values of two or more stimulation parameters.

The patient physiological response 2012 may include information of body, sites (response sites) where the effects of the first neuromodulation energy is perceived or otherwise detected such as by a sensor. The patient physiological response 2012 may be represented by texts, graphs, verbal description, among other means of representation. In an example, the patient physiological response 2012 may include a response map of the body sites responsive to the applied first neuromodulation energy, such as the example illustrated in FIG. 19. In some examples, the response map may include information of quality or intensity of the induced sensory response or the physiological response.

The patient physiological response 2012 may include one or more of a sensory input 2013 and physiological data 2014. The sensory input 2013 represents induced patient sensory feedback such as paresthesia. In an example, the sensory input 2013 may include a paresthesia drawing of body sites experiencing paresthesia (paresthesia sites) responsive to the applied first neuromodulation energy. The paresthesia drawing, such as produced by the patient, may include paresthesia markings that identify the paresthesia sites, and optionally paresthesia scores to Characterize intensities or qualities of paresthesia perception at each marked paresthesia site. The paresthesia scores may have categorical or numerical values representing a degree of patient preference or a degree of side effect of the induced paresthesia. For example, on a scale of 0-5, a paresthesia score of 0 indicates least preferred or a strongest side effect, and a paresthesia score of 5 indicates most preferred or a minimal side effect.

The physiological data 2014 may include a sensor data map representing sensor data across one or more body sites of the patient. The sensor data map may additionally include physiological response scores to characterize intensities or qualities of sensor signals acquired at the response sites. The physiological response scores may have categorical values or numerical values such as based on a comparison of a sensor signal metric (e.g., an amplitude, a frequency, or signal power) to one or more threshold values. For example, on a scale of 0-5, a physiological response score of zero indicates no response (e.g., a sensor signal amplitude below a low threshold), and a response score of five indicates a strongest physiological response (e.g., a sensor signal amplitude exceeding a high threshold).

The sensors used for sensing patient physiological response to the applied first neuromodulation energy (e.g., diagnostic neurostimulation for pain targeting) may be external or subcutaneous sensors. In an example, paresthesia sites are first identified, and one or more sensor are positioned at the identified paresthesia sites. In another example, one or more sensors may be positioned at one or more pain sites prior to delivery of the first neuromodulation energy. The sensors may pick up patient physiological responses from the pain-paresthesia overlapping areas. In some examples, one or more sensors are implantable sensors, such as associated with a neuromodulation lead or a separate implantable device, placed over regions of spinal cord, dorsal column, or dorsal roots, among other neural elements.

Examples of the sensors may include electromyography (EMG) sensors, electrospinogram (ESG) sensors, evoked compound action potential (eCAP) sensor, or impedance sensors, among others. The EMG sensors may be patches configured for external placement over the skin area experiencing paresthesia. In an example, the EMG sensors may be bilaterally placed (left and right), and/or placed on both the front and back of the patient. The EMG sensors may be implantable sensors such as associated with the implantable lead. The EMG signals may be recorded and processed, and one or more EMG metrics such as amplitude, timing (e.g., onset timing with respect to stimulation pulse), or frequency or spectral content, may be generated. The EMG metrics may be absolute values, or relative values such as between different EMG electrodes, between different dermatomes, or between left and right sides or between front and back of the patient. In another example, the ESG sensors can sense electrical activity (e.g., electrical field potential) of the spinal cord or the affiliated neural elements such as dorsal roots or spinal nerves. In yet another example, the eCAP sensors can sense a change in eCAP produced by the diagnostic electrostimulation. The ESG or the eCAP sensors may be placed on the paresthesia sites, or implanted such as associated with a neuromodulation lead or as a separately implanted device. In another example, one or more impedance sensors can sense tissue impedance at the pain sites or paresthesia sites. Changes in tissue impedance is correlated with paresthesia and muscle activities produced by the diagnostic electrostimulation.

Other sensors may additionally or alternatively be used, including, for example, a photoplethysmography (PPG) sensor, a near infrared spectroscopy (MRS) sensor, a Doppler ultrasound sensor, an accelerometer sensor, among others. A PPG sensor may utilize an infrared light to measure the volumetric variations of blood circulation. In an example, a PPG sensor may use an infrared light emitting diode (IR-LED) or a green LED as the main light source. Other light sources such as yellow LED may also be used. MRS is a non-invasive technique that allows determination of tissue oxygenation based on spectro-photometric quantitation of oxy- and deoxyhemoglobin within a tissue. A doppler ultrasound is a noninvasive test that can be used to estimate the blood flow through your blood vessels by bouncing high-frequency sound waves (ultrasound) off circulating red blood cells. A doppler ultrasound can estimate how fast blood flows by measuring the rate of change in its pitch (frequency). An accelerometer is a sensor for measuring acceleration, which can be used to measure the effects of muscle activation or blood flow changes. Any of these sensors can be used like the EMG sensors described. For example, they may be in an array of sensors that are in a patch that is adhered to the patient's skin that overlaps the region of pain. These sensors can be used to optimize the overlap of paresthesia with the region of pain.

The processor circuit 2030 is configured to determine an optimal stimulation field definition. The stimulation field definition includes a stimulation setting such as central point of stimulation (CPS) representing a focal point of a stimulation field, one or more stimulation parameters (also referred to as a neuromodulation parameter set) such as a current amplitude or a voltage amplitude, a pulse width, a pulse shape (waveform), a pulse rate, or a duty cycle, among others. The processor circuit 2030 may include circuit sets comprising one or more other circuits or sub-circuits, such as a dermatomal coverage analyzer 2031, an electrode configuration circuit 2032, and a stimulation controller 2033. The circuits or sub-circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

In various examples, portions of the functions of the processor circuit 2030 may be implemented as a part of a microprocessor circuit. The microprocessor circuit can be a dedicated processor such as a digital signal processor, application specific integrated circuit (ASIC), microprocessor, or other type of processor for processing information including physical activity information. Alternatively, the microprocessor circuit can be a general purpose processor that can receive and execute a set of instructions of performing the functions, methods, or techniques described herein.

The dermatomal coverage analyzer 2031 is configured to generate a pain targeting metric (PTM) using the pain data 2011 and the physiological response 2012 (e.g., one or both of the sensory input 2013 or the physiologic data 2014). The PTM represents a spatial correspondence between the pain sites and the body sites responsive to the applied first neuromodulation energy over one or more dermatomal compartments. A dermatome, or a dermatomal compartment, is an area of skin that is supplied by sensory neurons that arise from a spinal nerve ganglion. Originating from the spinal cord there are eight cervical nerves (C1 through C8), twelve thoracic nerves (T1 through T12), five lumbar nerves (L1 through L5), and five sacral nerves (S1 through S5). Each of these nerves relays sensations, including pain, from a particular region of skin to the brain. Successful pain management and the avoidance of stimulation in unafflicted regions require the applied electric field to be properly positioned longitudinally along the dorsal column. The spinal nerves (including cervical nerves C1-C8, thoracic nerves T1-T12, lumbar nerves L1-L5, and sacral nerves S1-S5) can be mapped to respective dermatomes. The dermatomes C1-S5 may be mapped to respective dermatome numbers (d), as illustrated in Table 1. More superior spinal nerves are mapped to smaller dermatome numbers, and more inferior spinal nerves are mapped to larger dermatome numbers. The dermatome number can be used to characterize the spatial extent of pain.

TABLE 1

| | Dermatome Number |
|---|---|
| Dermatome | (d) |
| C1 | 1 |
| C2 | 2 |
| . . . | . . . |
| C8 | 8 |
| T1 | 9 |
| T2 | 10 |
| . . . | . . . |
| T12 | 20 |
| L1 | 21 |
| L2 | 22 |
| . . . | . . . |
| L5 | 25 |
| S1 | 26 |
| S2 | 27 |
| . . . | . . . |
| S5 | 30 |

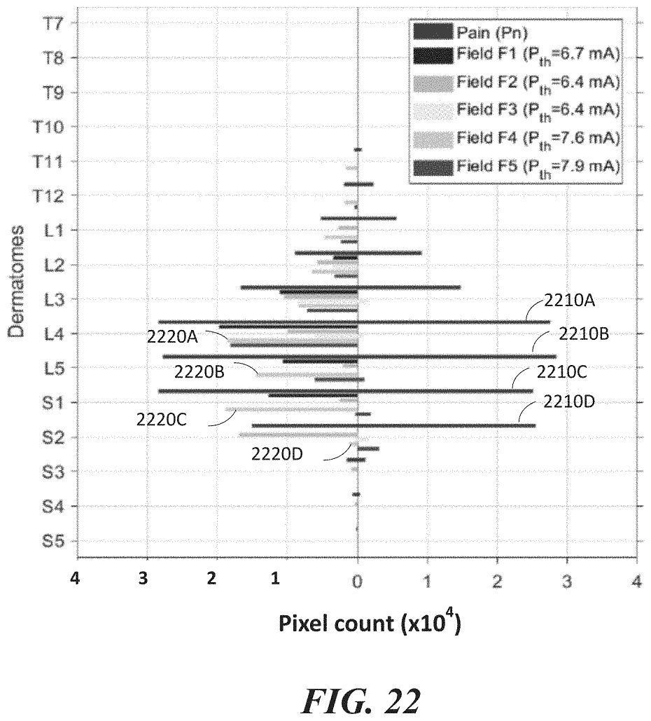

The PTM may be calculated as an overlapping area between a pain drawing (an example of the pain data 2011) and a paresthesia drawing (an example of the sensor input 2013) across one or more dermatomal compartments, or an overlapping area between the pain drawing and a sensor data map (an example of the physiological data 2014) across one or more dermatomal compartments. In some examples, the dermatomal coverage analyzer 2031 may determine a pain distribution across a first set of dermatomal compartments using the pain data 2011, and determine a response distribution (such as a paresthesia distribution, or a sensor data distribution) across a second set of dermatomal compartments using the received physiological response 2012. The pain distribution and the response distribution may each be computed using spatial information (e.g., pain sites, paresthesia sites, or sensor response sites), intensity information (e.g., pain intensity, paresthesia intensity, or sensor data intensity), or a combination of the spatial and intensity information. A PTM may be calculated using the pain distribution and the response distribution. Examples of the pain distribution, the response distribution, and the PTM are discussed below, such as with reference to FIG. 22.

The electrode configuration circuit 2032 may determine an optimal stimulation field definition using one or more PTMs from the dermatomal coverage analyzer 2031. The stimulation field definition includes stimulation parameters such as field size, shape, and intensity, field scaling and steering parameters, among others. The PTM, which is indicative of spatial correspondence between the pain perception and the paresthesia or physiological responses to diagnostic electrostimulation, may be used to determine for each electrode a corresponding anodic weight and a cathodic weight. The electrode has a net anode effect if the anodic weight is greater than the cathodic weight, or a net cathode effect if the anodic weight is less than the cathodic weight. A normalization factor may be applied to control a relative strength (proportion) of the anode effect or cathode effect distributed to that electrode. The normalization factors of all the electrodes with a net anode effect add up to 100%. The normalization factors of all the electrodes with a net cathode effect add up to −100%.

Based on the optimal stimulation field definition, the electrode configuration circuit 2032 may regulate neuromodulation energy (e.g., electrical current) to individual ones of a set of electrodes, a process referred to as energy or current fractionalization among electrodes, or fractionalized electrode configuration. In an example, the electrode configuration circuit 2032 may determine current fractionalization among multiple electrodes in accordance with an objective function. An objective function refers to a function with desirable characteristics for modulating the targeted tissue. The objective function may also be referred to as an objective target function. An objective function for a broad and uniform modulation field is identified for a given volume of tissue. Examples of an objective function includes a constant E (electric field), a constant E (electric field magnitude), and a constant voltage. Information such as lead and electrode configuration, electrode tissue coupling, electrode contact status, and a threshold such as a current threshold, may be used as input to an electrode energy fractionalizer that regulates electrical current to individual electrodes based on the stimulation field definition. A function can be performed that is dependent on the objective function, the electrode positions, and the electrode tissue coupling. The result of the function is the fractionalization of modulation energy (e.g. current) for each electrode to achieve the objective function. The fractionalization of modulation energy may be expressed, for each electrode, as a polarity and percentage of the total cathodic energy (add up to 100%) or percentage of total anodic energy (add up to −100%) delivered to the plurality of electrodes on the lead at a given time. Furthermore, an amplitude boost or scaling factor may be applied to the fractionalization values. In some examples, a selected field model may be used to estimate the field induced by unit current from the contact. The field is calibrated using the threshold. Constituent forces are formed based on the selected contacts. A transfer matrix (A) may be constructed, which mathematically describe the electrical behavior of the model. A specified target stimulation field may be provided to the model. The target stimulation field (φ) can be represented by a central point of stimulation (CPS). The transfer matrix (A) can be used to compute the minimal mean square solution using contributions from the constituent sources and the specified target field. The solution can be used to compute the current fractionalization on each contact. Electrodes configured in accordance with the current fractionalization may be used to establish a stimulation field to modulate target neural element for pain relief.

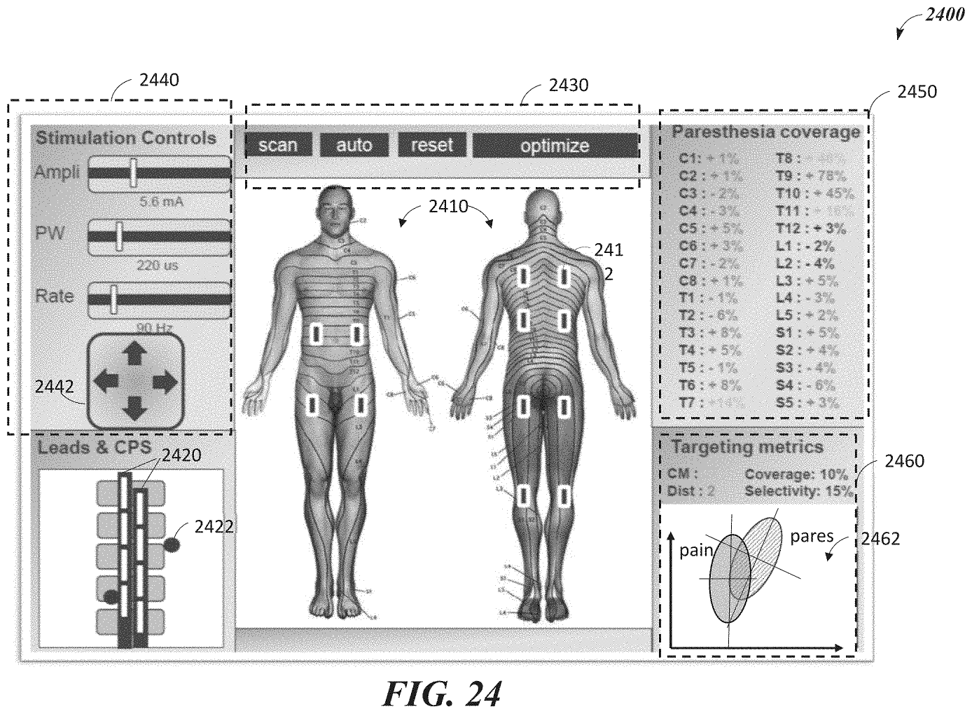

Information of electrode configuration, such as the optimal stimulation field definition and electrode fractionalization, may be presented to a user or a process. In an example, the output unit 2050 may include a user interface, such as one of the GUI 214 or 414 of the programming device 213, 413, or 628. The user interface may be communicatively coupled to the processor circuit, and configured to display the information of electrode configuration, among other information such as the pain distribution, the response distribution (e.g., paresthesia distribution or sensor data distribution), or the PTM. In an example, the user interface may receive user input for selecting a stimulation setting from a set of setting candidates (e.g., candidate stimulation fields), or for programming a stimulation setting such as by adjusting a value of one or more stimulation parameters. Example of a user interface for programming neuromodulation with interactive display of patient response and dermatomal coverage are discussed below, such as with reference to FIG. 24.

The stimulation controller 2033 may generate a control signal for adjusting a stimulation setting, such as by trolling the CPS location or tuning one or more stimulation parameters (e.g., pulse width, amplitude, duty cycle, stimulation rate, etc.). The control signal may trigger the electrostimulator 2040 to deliver first neuromodulation energy (diagnostic stimulation) to a target neural element to induce patient sensory feedback such as paresthesia, or to elicit a physiological response, which can be used for pain targeting. Additionally, the stimulation controller 2033 may generate a control signal that triggers the electrostimulator 2040 to deliver a second neuromodulation energy, such as sub-perception stimulation energy, at a target neural element to relieve pain via set of electrodes in accordance with the fractionalized electrode configuration determined by the electrode configuration circuit 2032. The sub-perception stimulation energy may be delivered using monophasic stimulation pulses applied to each electrode, which can be used as either an anode or cathode in accordance with the fractionalization configuration. Alternatively, the sub-perception stimulation energy may be delivered using biphasic stimulation pulses. Each biphasic pulse has a first phase of a first polarity followed by a second phase of a second polarity opposite of the first polarity. The first and second phases can be symmetric (e.g., the same magnitude or duration). Alternatively, the first and second phases can be asymmetric. Because of opposite polarities of the two phases in a stimulation pulse, the electrode polarity (i.e., designation of an electrode as an anode or as a cathode) as defined by the electrode configuration circuit 2032 would flip when the stimulation current changes from the first phase to the second phase. In an example, the electrode configuration circuit 2032 may modify the cathode and anode designation based on the first phase of the biphasic pulse. For example, if the first phase is positive, then no modification is made to the anode and cathode that have been determined. If the first phase is negative, then the anode and cathode designation are swapped. In an example, asymmetric biphasic stimulation may be delivered to both a preferred stimulation site and a site where stimulation is to be minimized. At the preferred stimulation site, the biphasic stimulation can include a first anodic phase with a longer duration and smaller magnitude, followed by a second cathodic phase with a shorter duration and larger magnitude. At the region to avoid, biphasic stimulation can include a first cathodic phase with a longer duration and smaller amplitude (sub-threshold for its pulse width), followed by a second anodic phase with a shorter duration and higher amplitude for charge balance.

The electrostimulator 2040 can be an implantable module, such as incorporated in the implantable system 521. Alternatively, the electrostimulator 2040 can be an external stimulation device, such as incorporated in the external system 522. In an example, the first and second neuromodulation energy may be delivered respectively through separate first and second channels of the electrostimulator 2040. Alternatively, the first and second neuromodulation energy may be generated and delivered respectively by different electrostimulator. For example, an external electrostimulator is used to generate the first neuromodulation energy to induce paresthesia or to elicit physiological response, and an implantable electrostimulator is used to generate the second neuromodulation energy as a mode of pain therapy.

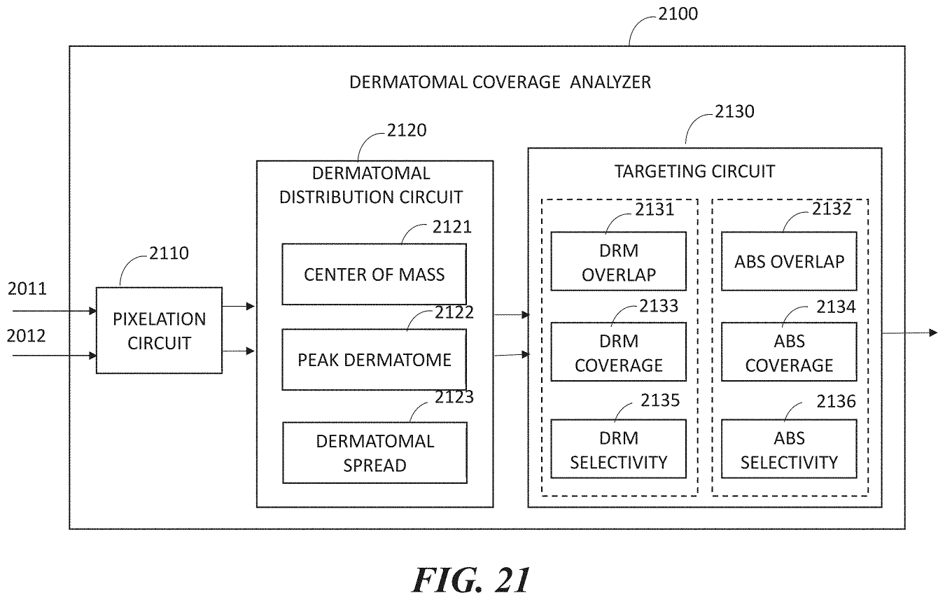

FIG. 21 is a block diagram illustrating, by way of example and not limitation, a dermatomal coverage analyzer 2100 configured to generate a pain targeting metric (PTM). The PTM represents a spatial correspondence between pain sites (S_{P}) and body sites responsive to the applied first neuromodulation energy (response sites S_{R}) over one or more dermatomal compartments. As discussed above, the response sites S_{R} can be paresthesia sites marked by the patient, or sensor response sites automatically detected by one or more sensors (e.g., an EMG sensor, an ESG sensors, an eCAP sensor, a PPG sensor, a MRS sensor, a doppler flowmetry sensor, an accelerometer sensor, or a gyroscope sensor). The dermatomal coverage analyzer 2100, which is an embodiment of the dermatomal coverage analyzer 2031 shown in FIG. 20, may include one or more of a pixelation circuit 2110, a dermatomal distribution circuit 2120, and a targeting circuit 2130. In various examples, some of all of circuits 2110, 2120, and 2130 may be implemented in a microprocessor configured to execute instructions to perform respective functions, methods, or techniques described herein.

The pixelation circuit 2110 may pixelate a pain drawing from the received pain data 2011 into pixels corresponding to anatomical point locations of the pain sites S_{P}. The point location of each pixel may be represented by pixel coordinates. In some examples, the pain drawing may include pain scores characterizing the pain intensity or pain quality at each pain site. Accordingly, the pixels of the pixelated pain drawing may each be associated with a pain descriptor indicating presence or absence, intensity (e.g., pain score), or temporal pattern of pain.

The pixelation circuit 2110 may similarly pixelate a response map, such as a paresthesia drawing from the sensor input 2013 or a sensor data map from the physiological data 2014, into pixels corresponding to anatomical point locations of the response sites S_{P}. The point location of each pixel may be represented by pixel coordinates. In some examples, the paresthesia drawing may include paresthesia scores representing patient preference or side effects of the paresthesia. Pixels of the pixelated paresthesia drawing are each associated with a paresthesia descriptor indicating presence or absence, intensity (e.g., paresthesia score), or temporal pattern of paresthesia at the corresponding pixel. Likewise, in those examples where the sensor data map includes physiological response scores, pixels of the pixelated sensor data map may each be associated with a physiological response descriptor indicating presence or absence, signal intensity (e.g., sensor signal amplitude), or temporal pattern of physiological response at the corresponding pixel.

The dermatomal distribution circuit 2120 may map the pain sites S_{P} to a first set of dermatomal compartments {d_{P}}, which is a subset of the dermatome numbers 1-30 as illustrated in Table 1. The dermatomal distribution circuit 2120 may determine a pain distribution over the dermatomal compartments {d_{P}} using the pixelated pain drawing, and calculate one or more dermatomal metrics from the pain distribution. FIG. 21 illustrates non-limiting examples of dermatomal metrics, such as a center of mass 2121, a peak dermatome 2122, or a dermatomal spread 2123. The center of mass 2121, denoted by CM, is a focal point representing the center of pain effects across the dermatomal compartments {d_{P}}. In an example, the center of mass 2121 may be determined using a weighted sum of the dermatomal compartments in the set {d_{P}}, as given in Equation (1):

CM=Σᵢkᵢ*dᵢ (1)

where the summation is over all dermatomal compartments dᵢ that belong to {d_{P}}.

In some examples, the weight factor kᵢ for the dermatomal compartment dᵢ may be proportional to dᵢ's contribution to the overall pain effect across all the dermatomal compartments {d_{P}}, such as given by:

kᵢ=x_{d}_{ᵢ}/Σᵢx_{d}_{ᵢ}

where x_{di} is a dermatome-level pain effect of the dermatome dᵢ, and the sum Σᵢx_{d}_{ᵢ} represents the overall pain effect across {d_{P}}. By plugging kᵢ into Equation (1), the center of mass 2011 may be computed using Equation (2):

CM=(Σᵢx_{d}_{ᵢ}*dᵢ)/Σᵢx_{d}_{ᵢ} (2)

In an example where each pixel is associated with a binary pain value (0 for absence, and 1 for presence, of pain at that pixel location), the dermatome-level pain effect x_{di} can be determined by counting the number of pixels in the dermatomal compartment dᵢ with a pain value of 1. For example, a pain drawing is mapped to three dermatomal compartments T12 (d=20), L1 (d=21), and L2 (d=22), and the pixelation process indicates that the numbers of pixels with a pain effect equal to one (pain present) in those dermatomal compartments are: 100 in T12, 200 in L1, and 250 in L2. Then, the center of mass may be determined using Equation (2):

CM=(100*20+200*21+250*22)/(100+200+250)=21.3

The CM may be rounded to the nearest integer dermatome 21, which corresponds to dermatomal compartment L1, according to Table 1. In an example where each pixel has a discrete pain value representing a discrete pain intensity level (e.g., on a scale of 0-5), the dermatome-level pain effect x_{di} may be computed using a cumulative pain value over all the pixels in the dermatomal compartment dᵢ.

The peak dermatome 2122, denoted by dMax, represents a dermatomal compartment that has a largest dermatome-level pain effect among {d_{P}}, as given in Equation (3):

dMax=argmax_{d}_{ᵢ}−{_{d}_{ᵢ}}, for dᵢ∈{dₚ} (3)

In an example where each pixel is associated with a binary pain value, the dMax represents the dermatomal compartment that encompasses the largest number of non-zero pixels, thus the broadest pain coverage, among all dermatomes in {d_{P}}. In the above example that involves T12, L1, and L2, dMax is determined to be L2, as L2 encompasses the largest number of non-zero pixels.

The dermatomal spread 2123, denoted by Φ, represents a subset of {d_{P}} that have a dominant dermatome-level pain effect in the pixelated pain drawing. In an example of binary pain value at the pixel level, the dermatomal spread 2123 may be determined as a range of dermatomal compartments with a cumulative count of non-zero pixels amounting to at least a specified fraction (e.g., approximately 75%) of the total non-zero pixels in {d_{P}}. In an example of discrete pain values at the pixel level, the dermatomal spread 2123 may be determined as a range of dermatomal compartments with cumulative pain values exceeding a specified fraction (e.g., 75%) of the sum of pain values of all the pixels in {d_{P}}.

In an example, the dermatomal spread 2123 may be computed using the center of mass 2121 (CM), as given in Equation (4):

$$Φ = \sqrt{\frac{{Σ}_{i} {x}_{{d}_{i}} * {( {d}_{i} - CM )}^{2}}{( {Σ}_{i} {x}_{{d}_{i}} ) - {x}_{{d}_{CM}}}} \qquad ( 4 )$$

The center of mass may be computed using Equation (2) above. The term (dᵢ−CM) represents the deviation of the dermatomal compartment dᵢ from the center of mass, and x_{dCM} represents the dermatome-level pain effect at the dermatome at, or closest to, the center of mass (i.e., d=CM). In the above example that involves T12, L1, and L2, the center of mass is CM=21.3, x_{dCM}=x_{L21}=200, so the spread is:

Φ=(100*(20−21.3)²+200*(21−21.3)²+250*(22−21.3)²)/(100+200+250−200)=0.88.

The dermatomal distribution circuit 2120 may similarly map the response sites S_{R} to a second set of dermatomal compartments {d_{R}}, also a subset of the dermatome numbers 1-30 as illustrated in Table 1. The dermatomal distribution circuit 2120 may generate a response distribution of the pixelated response map (e.g., a pixelated paresthesia map, or a pixelated sensor data map) across the dermatomal compartments {d_{R}}. One or more of the dermatomal metrics discussed above, such as center of mass 2121, the peak dermatome 2122, or the dermatomal spread 2123, may be used to quantify the response distribution. For example, the center of mass 2121 of the pixelated response map may be calculated using the dermatome-level physiological responses across the dermatomal compartments {d_{R}}, according to Equations (1) or (2). The peak dermatome 2122 that has a largest dermatome-level physiological response among the dermatomes in {d_{R}} may be determined according to Equation (3). The dermatomal spread 2123 may be determined as a subset of {d_{R}} that have a dominant dermatome-level physiological response, such as exceeding a threshold fraction (e.g., approximately 75%) of the sum of physiological responses of all the pixels in {d_{R}}. In an example, the dermatomal spread 2123 of the physiological responses may be computed according to Equation (4).

The targeting circuit 2130 may be configured to generate a pain targeting metric (VIM) using the pain distribution and the response distribution. The PTM represents a spatial correspondence between the pain sites and the response sites. In an example, the VIM may include a dermatomal overlap 2131, denoted by PRo, which represents an overlap between the pain sites and response sites. The dermatomal overlap 2131 may be computed using the overlap between the dermatome-level pain effect x_{di} and the dermatome-level physiological response y_{di}, accumulated over all the dermatome compartments (or at least the union of the sets {d_{P}} and {d_{R}}), as given in Equation (5):

PRo=Σᵢ min(x_{d}_{ᵢ},y_{di}) (5)

where min (x_{di}, y_{di}) represents the overlap between the dermatome-level pain effect and the dermatome-level physiological response at dermatomal compartment dᵢ. In an example, the physiological response y_{di} may be represented by categorical values over a range of value grades. In an example, the dermatomal overlap 2131 may be computed based on a comparison of a peak value of dermatome-level pain effect across a range of dermatome compartments and a peak physiological response value across the same range of dermatome compartments.

In an example, the PTM may include a dermatomal coverage 2133, denoted by PRc, representing a relative amount (e.g., a fraction) of the pain sites that are mapped to the response sites where paresthesia is induced or physiological responses are elicited and detected by a sensor. In an example, the PRc may be computed using a ratio of the dermatomal overlap PRo to a sum of the dermatome-level pain effects over all the dermatomes in {d_{P}}, as given in Equation (6):

PRc=PRo/Σᵢx_{d}_{ᵢ} (6)

In an example, the PTM may include a dermatomal selectivity 2135, denoted by PRs, representing a relative amount (e.g., a fraction) of the response sites that overlap with the pain sites. In an example, the PRs may be computed using a ratio of the dermatomal overlap PRo to the sum of the dermatome-level physiological responses over all the dermatomes in {d_{R}}, as given in Equation (7):

PRs=PRo/Σᵢy_{d}_{ᵢ} (7)

The PRs is an indicator of efficiency of the diagnostic stimulation programmed with a particular stimulation setting in targeting the pain sites. For example, if a diagnostic stimulation, in accordance with a first stimulation setting, results in a large PRs value, it indicates that the induced paresthesia or elicited physiological responses are mostly located within the pain sites, and the diagnostic stimulation is therefore efficient in pain targeting. In contrast, if a diagnostic stimulation in accordance with a second stimulation setting results in a small PRs value, it indicates that said stimulation has induced paresthesia or elicited physiological responses in a large body area outside the pain sites. Such diagnostic stimulations is therefore less efficient in targeting the pain.

The dermatomal overlap 2131, the dermatomal coverage 2133, and the dermatomal selectivity 2135 are dermatome-based metrics derived from the pain distribution and the response distribution over respective multiple dermatomal compartments. Alternatively, the pain distribution and the response distribution may be based on pixel coordinates, and pixel coordinates-based metrics (also referred to as absolute metrics) may be derived from respective distributions, including one or more of an absolute overlap (PRo′) 2132, an absolute coverage (PRc′) 2134, or an absolute selectivity (PRs′) 2136. The PRo′ 2132 may be determined using an intersection, such as a count of overlapped pixels, between the pixelated pain drawing (X_{i,j}) the pixelated response map (Y_{i,j}), as given in Equation (8):

PRo′=X_{i,j} [symbol] Y_{i,j} (8)

The PRc′ 2134 can be determined using a ratio of the count of overlapped pixels PRo′ to a count of pixels in the portion of the pixelated pain drawing corresponding to the first set of dermatomal compartments {d_{P}}. A method of computing the PRc′ 2134 is given in Equation (9). Similar to the PRc 2133, the PRc′ 2134 may be represented fraction of the pain area targeted by paresthesia perception or physiological response sensing through diagnostic stimulation in accordance with a particular stimulation setting.

PRc′=PRo′/Σ_{i,j}X_{i,j} (9)

The PRs′ 2136 can be determined using a ratio of the count of overlapped pixels PRo′ to a count of pixels in the portion of the pixelated response map corresponding to the second set of dermatomal compartments {d_{R}}. A method of computing the PRs′ 2136 is given in Equation (10). Similar to the PRs 2135, the PRs′ 2136 indicates an efficiency of the diagnostic stimulation programmed with a particular stimulation setting in targeting pain.

PRs′=PRo′/Σ_{i,j}Y_{i,j} (10)

In various examples, a PTM, such as the dermatomal overlap 2131, may be determined using one or more of the center of mass 2121, the peak dermatome 2122, or the dermatomal spread 2123 respectively computed for the pain drawing across the dermatomal compartment set {d_{P}} and for the response map across the dermatomal compartment set {d_{R}}. In an example, the dermatomal overlap 2131 may be determined using a relative difference between the center of mass of the pixelated pain drawing (CM_{P}) and the center of mass of the pixelated response map (CM_{R}). In another example, the dermatomal overlap 2131 may be determined using a relative difference between the peak dermatome (dMax_{P}) having a largest dermatome-level pain effect among the dermatomal compartments {d_{P}}, and the peak dermatome (dMax_{R}) having a largest dermatome-level physiological response among the dermatomal compartments {d_{R}}. In yet another example, the dermatomal overlap 2131 may be determined using a relative difference between the dermatomal spread (Φ_{P}) of the pixelated pain drawing and the dermatomal spread (Φ_{R}) of the pixelated response map. The PTM may be determined using one or more dermatomal overlap 2131 computed using the approaches as discussed above. In an example, the PTM includes a linear or nonlinear combination of multiple dermatomal overlap metrics, such as given in Equation (11):

PTM=a*|CM_{P}−CM_{R}|+b*|dMax_{P}−dMax_{R}|+c*|Φ_{P}−Φ_{R}| (11)

where a, b, c are scaling factors.

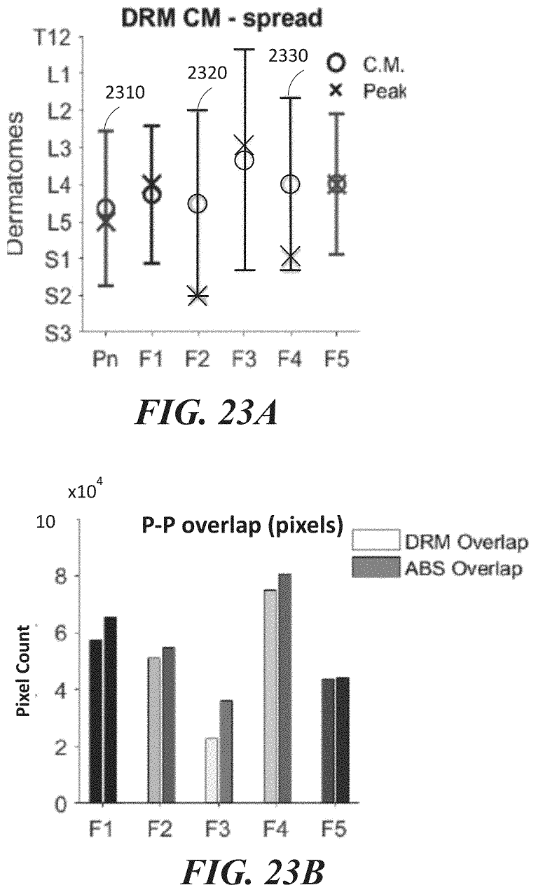

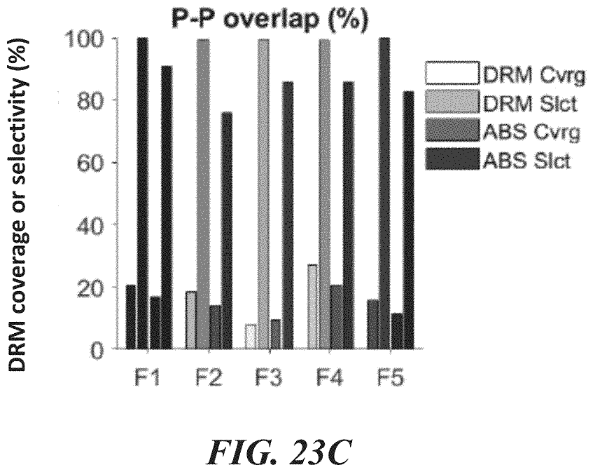

The PTMs generated by the targeting circuit 2130 may then be used by the electrode configuration circuit 2032 to determine an optimal stimulation field definition and to fractionalize current among the electrodes. Examples of pain distribution or response distribution, and various metrics such as center of mass, peak dermatome, dermatomal spread, dermatomal or absolute overlap, coverage, and selectively are discussed below, such as with reference to FIGS. 22, 23A-23C.

FIG. 22 is a diagram illustrating, by way of example and not limitation, a pain distribution, and a plurality of paresthesia distributions corresponding to different stimulation fields. The pain distribution may be generated from a pain drawing, such as produced by a patient and received from the data receiver 2011. The plurality of paresthesia distributions are obtained from respective paresthesia drawings or sensor data maps obtained from the same patient during or after the application of diagnostic stimulation in accordance with respective stimulation settings. The pain drawing and the plurality of paresthesia drawings may each be mapped to the multiple dermatomal compartments.

The pain distribution and the plurality of paresthesia distributions are represented as pixel counts (on the x-axis) across multiple dermatomal compartments (on the y-axis). By way of example and not limitation, pixels that fall into one side of dermatome (e.g., a dermatome portion on the left side of patient body) and pixels that fall into an opposite side of the dermatome (e.g., a dermatome portion on the right side of patient body) may be counted separately. The illustrated example shows, for each dermatome, a first pixel count on one side of patient body (left of “0” on the x-axis) and a second pixel count on the opposite side of patient body (right of “0” on the x-axis). Plotted along with the pain distribution are five paresthesia distributions, represented by pixel within each of the dermatomal compartments. Said paresthesia distributions correspond to paresthesia-induction stimulations in accordance with respective stimulation fields, F1-F5. The stimulation fields F1-F5 differ from one another by at least one stimulation parameter e.g., CPS, pulse amplitude, pulse width, pulse rate, or pulse waveform, among others). Electrostimulation delivered according to F1-F5 may induce paresthesia at different dermatomal compartments, represented by pixel counts from the pixelated paresthesia drawing.

As illustrated in FIG. 22, the pain distribution spreads over a range of dermatomes from T11 to S4. The pixels are predominantly distributed in L4, L5, and S1 dermatomes, with corresponding pixel counts 2210A, 2210B, and 2210C. In each of these dermatomes, the pixel counts on both sides of the patient body (left and right of “0” on the x-axis) are approximately the same (between 25,000-30,000 pixels on either side), indicating symmetric pain areas at these dermatome levels. At S3, more pixels are located on the right side than on the left site, indicating a non-symmetric pain distribution that is biased towards the right side of the body.

The paresthesia distributions corresponding to the stimulation fields F1-F5 may each spread over respective ranges of dermatomes, and predominant pixels counts are located at some dermatomes. By way of example, stimulation in accordance with F4 induces paresthesia, and the resultant paresthesia distribution shows predominant pixel counts 2220A, 2220B, and 2220C found in dermatomes L4, L5, and S1, the same dermatomal compartments where the predominant pain pixels counts are located. However, substantially less pixels 2220D are found at S2, indicating mild paresthesia perception at S2 level. Therefore, stimulation in accordance with F4 has an inferior pain targeting performance at S2 level than at L4, L5, or S1 levels. Based on the pain distribution and the paresthesia distributions shown in FIG. 22, one or more dermatomal metrics, such as a center of mass 2121, a peak dermatome 2122, or a dermatomal spread 2123, may be derived respectively from the pain distribution, and each of the five paresthesia distributions, such as by using Equations (1)-(4), as discussed above with reference to FIG. 21. In some examples, the diagram of FIG. 22 or a variant thereof, or information about the pixel counts at each dermatome, may be displayed to a user such as via the output unit 2050.

In the illustrate example shown in FIG. 22, pixels of the pain drawing and the paresthesia drawings each has a binary pain value representing presence or absence of pain at that pixel location. The dermatome-level pain effect and the dermatome-level physiological response (paresthesia effects such as patient preference or side-effect scores) at a particular dermatome is represented by the pixel count at said dermatome. In some examples, pixels of the pain drawing and the paresthesia drawings each has a discrete pain value (such as on a scale of 0-5) representing a discrete level of pain intensities or a discrete level of physiological responses. The distribution diagram FIG. 22 may be modified to represent, for each of multiple dermatomal compartments (on the y-axis), the dermatome-level pain effects (on the x-axis) calculated using a sum of pain values of all the pixels in a dermatomal compartment, and the dermatome-level physiological responses (on the x-axis) calculated using a sum of patient preference or side-effect scores of all the pixels in the dermatomal compartment.

FIGS. 23A-23C are diagrams illustrating, by way of example and not limitation, dermatomal metrics and pain targeting metrics (PTMs) computed using the pain distribution and the paresthesia distributions corresponding stimulation fields F1-F5, as shown in FIG. 22. FIG. 23A illustrates dermatomal metric plots depicting metrics of center of mass (CM), peak dermatome (dMax), and dermatomal spread (Φ), respectively derived from the pain distribution and the paresthesia distributions. The dermatomal metrics may be generated using the dermatomal distribution circuit 2120, such as according to Equations (1)-(4). For pain distribution (Pn) and each of the paresthesia distributions (F1-F5), the CM and dMax are marked by “0” and “X”, respectively, and the spread Φ is represented by a bar extending both directions from the CM. For the pain distribution, CM±Φ defines a range of dermatomal compartments where a dominant number of pixels from the pain drawing are located. Similarly, for a paresthesia distribution, CM±Φ defines a range of dermatomal compartments where a dominant amount of pixels from the paresthesia drawing are located. For example, the dermatomal metric plot 2310 of pain Pn shows that the CM of the pain distribution is located at approximately L5. Dominant pain perception spreads through L3, L4, L5, S1 and S2.

The paresthesia distributions may be compared to the pain distribution, and a PTM may be computed using one or more of the CM, the dMax, or the dermatomal spread Φ. In an example, the PTM may be calculated using Equation (11) for each of the paresthesia distributions. The stimulation field with the corresponding paresthesia distribution that yields the smallest PTM value can be identified and chosen as an optimal stimulation filed. In an example, a user (e.g., a clinician) may select an optimal stimulation field among F1-F5, such as via a user interface. For example, the stimulation field F2 corresponding to the dermatomal metric plot 2320, or F4 corresponding to the dermatomal metric plot 2330, may be decided as an optimal stimulation setting due to the closeness of the CM of the paresthesia distribution to the CM of the pain distribution, and that the spread of pain substantially, fall into the spread of said dermatomes. Other criteria based on one or more of CM, dMax, or Φ may be used to determine an optimal stimulation field.

FIG. 23B is a diagram illustrating an overlap between the pain drawing and each of the paresthesia drawings. Both the dermatomal overlap (PRo) 2131 as computed using Equation (5), and the absolute overlap (PRo′) 2132 as computed using Equation (8) are shown. In the illustrated example, among the stimulation fields F1-F5, F4 corresponds to the largest PRo and the largest PRo′, indicating that the stimulation programmed with F4 induces paresthesia that spatially overlaps with larger area of pain sites than stimulations programmed with other fields.

FIG. 23C is a diagram illustrating coverage and selectivity metrics between the pain drawing and each of the paresthesia drawings. The dermatomal coverage (PRc) 2133 is computed using Equation (6), and an absolute coverage (PRc′) 2134 is computed using Equation (9), The dermatomal selectivity (PRs) 2135 is computed using Equation (7), and the absolute selectivity (PRs′) 2136 is computed using Equation (10), Among the stimulation fields F1-F5, paresthesia drawing corresponding to F4 has the largest PRc (approximately 25%) and the largest PRc′ (approximately 20%), indicating that the stimulation programmed with F4 produces paresthesia that covers a larger percentage of pain area than stimulations programmed with other fields. With regard to selectivity, all paresthesia drawings have PRs at about 100%. Paresthesia drawings corresponding to F3 and F4 both have the largest PRs′ (approximately 85%) than other stimulation fields, indicating that the stimulation programmed with F3 or F4 would produce a smaller fraction of paresthesia area not overlapping with the pain area. Therefore, the stimulation programmed with F3 or F4 are more efficient in targeting the pain sites than stimulations programmed with other stimulation fields.

At least a portion of one or more of the diagrams shown in FIGS. 23A-23C may be displayed, such as via the output unit 2050. Additionally or alternatively, dermatomal metrics such as CM, dMax, and Φ for pain distribution and various paresthesia distributions, and PTMs such as dermatomal overlap, dermatomal coverage, or dermatomal selectivity, may be presented to a user (e.g., a clinician). The user may select an optimal stimulation field from candidate fields (e.g., F1-F5) such as via a user interface.

FIG. 24 is a diagram illustrating, by way of example and not limitation, a portion of a user interface 2400 for performing a pain-targeting test to find an optimal parameter setting. The user interface 2400 can be integrated into a neuromodulation programmer, such as the programming device 213 or 413, the CP 628, or the output unit 2050. The user interface can be an embodiment of the graphical user interface 414.

The user interface 2400 may include a display configured to show graphically or textually various dermatomal compartments on a body representation 2410 from different anatomical positions, such as an anterior (or ventral) position and a posterior (or dorsal) position as shown in FIG. 24. In some examples, locations of one or more sensors 2412 for collecting physiological data (e.g., EMG, ESG, PPG data, NIRS data, doppler flowmetry data, accelerometer sensor data, or gyroscope sensor data, etc.) may be shown on the body representation 2410. As previously discussed, the sensors may sense patient responses to a first neuromodulation energy. In an example, paresthesia drawing, including information of paresthesia sites, intensity, or quality of paresthesia perception at the paresthesia sites, may be marked on the body representation 2410. Similarly, patient pain drawing, including information of pain sites, intensity, or quality of pain at different pain sites, may be marked on the body representation 2410.

The display may additionally include a representation of one or more neuromodulation leads 2420 and portions of the environment in which the lead representation 2420 is placed, such as neural elements (e.g., spinal cord, spinal nerves, ventral or dorsal roots, etc.). In some examples, central point of stimulation (CPS) 2422 may be shown on top of the leads representation 2420. The CPS represents a locus of the first neuromodulation energy to induce paresthesia or to elicit physiological responses.

The user interface 2400 may include one or more user controls, such as system command controls 2430 and stimulation controls 2440, which are embodiments of user input device 1710 shown in FIG. 17. A user may use the one or more controls to program the electrostimulator (such as the electrostimulator 2040) to deliver the first neuromodulation energy for pain targeting, or to deliver the second neuromodulation energy for pain relief Some of the one or more controls may allow a user to adjust the view of the body presentation or the location of the sensor representation. As illustrated in FIG. 24, the system command controls 2430 may include mode selection control for selecting or deselecting a mode of pain targeting (a scan mode or an auto mode), a reset control for initiating or resetting the pain targeting operation, and an optimization control for optimizing a parameter setting based on dermatomal coverage analysis as discussed above with reference to FIGS. 20-22 and 23A-23C. The stimulation controls 2440 allow a user to adjust a stimulation parameter or to select a parameter from a list of values, for use in pain targeting. By way of example and not limitation, the stimulation parameters may include pulse amplitude, pulse width, pulse rate, among others. The stimulation controller 2033 may apply the programmed parameter values to the electrostimulator. In some examples, the stimulation controls 2440 may include a navigation control 2442 that allow a user to navigate through different display zones and control options. In some examples, the navigation control 2442 may allow a user to reposition the CPS 2422, such as by trolling the CPS 2422 up and down (i.e., rostrocaudally) along the lead representation 2420 and/or medic-laterally across the lead representation 2420. This would move the locus of the neuromodulation across different spinal cord locations or different neural elements. Trolling may be performed using a monopolar troll (e.g., anodic trolling or cathodic trolling), a bipolar troll, or a multipolar troll algorithm. A repositioned CPS, along with the stimulation parameter values programmed via the stimulation controls 2440, define a stimulation setting for inducing paresthesia or eliciting patient physiological response. In some examples, the navigation control 2442 may allow a user to reposition the sensor representation 2412 to different locations of the body representation.

The user interface 2400 can display results of pain targeting, such as one or more dermatomal metrics (e.g., CM, dMax, spread Φ), or one or more pain targeting metrics (PTMs, such as PRo, PRo′, PRc, PRc′, PRs, PRs′), as discussed above with reference to FIGS. 21, 22, and 23A-23C. In some examples, the user interface 2400 may include a display of one or more of the diagrams as illustrated in FIGS. 22 and 23A-23C. The result of pain targeting may be displayed or reported in formats of texts, graphs, or any combination thereof. In the example illustrated in FIG. 24, the pain targeting results include paresthesia coverage 2450 and targeting metrics 2460. The paresthesia coverage 2450 represents a change of paresthesia coverage before and after the application of the first neuromodulation energy. Referring now to FIG. 20, the processor circuit 2030 may generate a response map using physiological data collected during or after the application of the first neuromodulation energy, relative to baseline physiological data collected before the application of the first neuromodulation energy. The dermatomal coverage analyzer 2031 may determine the baseline PRc or the PRc′ prior to stimulation, and determine PRc or the PRc′ during and after the application of stimulation. The change of paresthesia coverage (ΔPRc, or ΔPRc′) from the baseline may indicate responsiveness of different dermatomal compartments to the stimulation and its special correspondence to the pain area at the corresponding dermatomal compartments.

The targeting metrics 2460 includes information of the dermatomal metrics and pain targeting metrics such as CM, distance (or “dist”, representing the number of dermatomes apart between the CM of pain distribution and the CM of physiological response distribution), dermatomal coverage, and dermatomal selectivity. The targeting metrics 2460 may additionally include a graph showing the spatial location of dominant pain area (such as based on the CM and spread Φ of the pain distribution) relative to the spatial location of dominant paresthesia area (such as based on the CM and spread Φ of the paresthesia distribution).

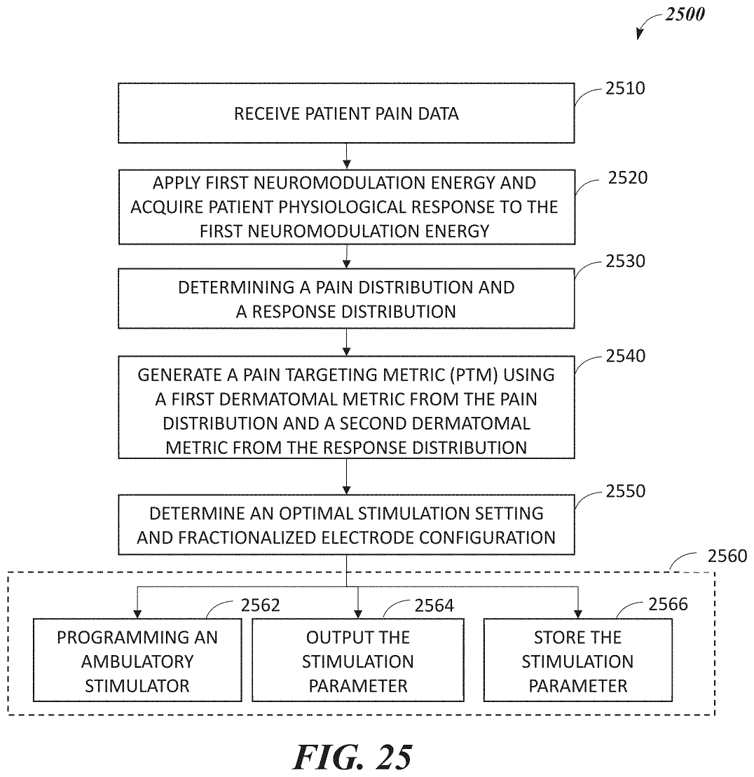

FIG. 25 is a flow chart illustrating, by way of example and not limitation, a method 2500 for optimizing neurostimulation for pain relief via a medical system or device, such as one of the neuromodulation systems 210 or 2000. Portions of the method 2500 may be implemented in an external device, such as a device in the external system 522, or one of the programming devices 213 or 413, the CP 628, or the RC 627. By executing the method 2500, the programming device or the external device may program a neuromodulation device (e.g., one of the neuromodulation devices 212, 312 or 512, or the IPG 626, or the electrostimulator 2040) to deliver neuromodulation energy, such as spinal cord stimulation (SCS). The method 2500 may be used to provide neuromodulation of other neural elements, such as one or more of spinal nerves, dorsal roots, or dorsal root ganglia, among others.

The method 2500 commences at 2510, where patient pain data can be received, such as from a user via the data receiver 2010. The pain data may include information of body sites of pain perception, and optionally pain characteristics such as pain intensity (e.g., categorical or numerical pain scores), pain quality (e.g., throbbing, stabbing, burning, pins and needles, crushing), or temporal pattern of pain. In an example, the pain data includes a pain drawing, such as the example illustrated in FIG. 19. The pain drawing may include pain markings, provided by the patient, that identify the locations where pain radiates or expands. The pain data may include reference data, such as a dermatome map that identifies various skin areas corresponding to spinal nerves that innervates said skin areas.

At 2520, a first neuromodulation energy is applied to a target neural element. The first neuromodulation energy may be generated and delivered to the target neural element by the electrostimulator 2040 or a different electrostimulator, according to a stimulation setting via a set of electrodes at respective electrode locations. The first neuromodulation energy can induce patient sensory feedback such as paresthesia, or elicit patient physiological response that can be detected by a sensor. Because the first neuromodulation energy can be used for targeting pain, it is also referred to as diagnostic stimulation in this document. In an example, the first neuromodulation energy includes supra-perception stimulation. The stimulation setting may include a central point of stimulation (CPS) representing a focal point of a stimulation field, a pulse amplitude, a pulse width, a pulse shape (waveform), a pulse rate, or a duty cycle, among other parameters.

Also at 2520, patient physiological response to the first neuromodulation energy is acquired. Such physiological response may include information of body sites (response sites) where the effects of the first neuromodulation energy is perceived by the patient, or detected by a sensor. The patient physiological response may include a response map of the body sites responsive to the applied first neuromodulation energy, such as the example illustrated in FIG. 19. In some examples, in addition to information of patient body sites that experience the induced sensory feedback (e.g., paresthesia) or the physiological response (e.g., sensor signals), the response map may include information of quality or intensity of the induced sensory feedback or the physiological response. In an example, the response map may include a paresthesia drawing, such as produced by that patient, that includes body sites experiencing paresthesia (paresthesia sites) responsive to the applied first neuromodulation energy, and optionally paresthesia scores representing a degree of patient preference or a degree of side effects of the induced paresthesia. In another example, the response map may include a sensor data map representing sensor data acquired from one or more body sites of the patient. In addition to the body sites information, the sensor data map may optionally include physiological response scores to characterize intensities or qualities of sensor signal at each sensor response site. The patient physiological responses may be sensed using a sensor or an array of sensors of the same or different modalities. Examples of the sensors include electromyography (EMG) sensors, electrospinogram (ESG) sensors, evoked compound action potential (eCAP) sensors, PPG sensors, NIRS sensors, doppler flowmetry sensors, accelerometer sensors, or a gyroscope sensors among others.

At 2530, a pain distribution across a first set of dermatomal compartments, and a response distribution (such as a paresthesia distribution, or sensor data distribution) across a second set of dermatomal compartments, may be determined respectively such as by using the dermatomal coverage analyzer 2031 or 2100. The pain distribution may be determined using spatial information (e.g., pain sites), intensity information (e.g., pain scores), or a combination of the spatial and intensity information from the received pain data. Similarly, the response distribution may be determined using spatial information (e.g., paresthesia sites or sensor response sites), intensity information (e.g., paresthesia scores such as patient preference or side effects, or sensor data intensity scores), or a combination of the spatial and intensity information from the received physiological data.

In an example, the received pain data includes a pain drawing. The pain drawing may be pixelated into pixels corresponding to anatomical point locations of the pain sites, such as via the pixelation circuit 2110. The pain sites may be mapped to a first set of dermatomal compartments NO In an example where the pixels of the pixelated pain drawing are each associated with a binary pain value (e.g., 0 for no pain and 1 for pain), the pain distribution may be represented by pixel counts across multiple dermatomal compartments, an example of which is illustrated in FIG. 22.

In an example, the received patient physiological response includes a response map, such as a paresthesia drawing or a sensor data map, corresponding to a particular stimulation setting (e.g., one of the stimulation fields F1-F5 as shown in FIG. 22). The response map may be pixelated into pixels corresponding to anatomical point locations of the response sites, such as via the pixelation circuit 2110. The response sites may be mapped to a second set of dermatomal compartments {d_{R}}. In an example where the pixels of the pixelated response map are each associated with a binary response (e.g., 0 for absence of paresthesia or no sensor response, and 1 for presence of paresthesia or positive sensor response), the response distribution may be represented by pixel counts across multiple dermatomal compartments, an example of which is illustrated in FIG. 22.

At 2540, a pain targeting metric (PTM) may be generated using the pain distribution and the response distribution. The PTM representing a spatial correspondence between the pain site and the body site responsive to the applied first neuromodulation energy at one or more dermatomal compartments. The PTM may be computed using one or more dermatomal metrics respectively derived from the pain distribution and from the response distribution, such as by using the dermatomal distribution circuit 2120. Examples of the dermatomal metrics may include a center of mass (CM), a peak dermatome (dMax), or a dermatomal spread (Φ). The CM, dMax, and Φ may be computed using Equations (2)-(4), respectively. The CM of the pain distribution is a focal point representing the center of pain effects across the dermatomal compartments {d_{P}}. The dMax of the pain distribution represents a dermatomal compartment having a largest dermatome-level pain effect among the dermatomal compartments {d_{P}}. The spread Φ of the pain distribution represents a subset of {d_{P}} that have a dominant dermatome-level pain effect in the pixelated pain drawing. Similarly, one or more dermatomal metrics, such as CM, dMax, and Φ may be similarly calculated for the response distribution, such as according to Equations (2)-(4), respectively. Examples of the said dermatomal metrics respectively derived from the pain distribution and the paresthesia distributions are illustrated in FIG. 23A.

Also at 2540, a PTM may be generated using one or more dermatomal metrics, such as CM, dMax, and Φ, respectively derived from the pain distribution and the response distribution, such as via the targeting circuit 2130. Examples of the PTM may include dermatome-based PTM, such as one or more of a dermatomal overlap PRo, a dermatomal coverage PRc, or a dermatomal selectivity Ms. The PRo represents an overlap between the pain sites and response sites. The PRc represents a fraction or a percentage of pain area that is mapped to the response sites where paresthesia is induced or physiological responses are elicited by a diagnostic stimulation. The PRs represents a fraction or a percentage of response sites that fall into the pain site, and indicates an efficiency of the diagnostic stimulation in targeting the pain sites. The PRo, PRc, and PRs may be computed using Equations (5)-(7), respectively. In some examples, the pain distribution and the response distribution may be based on pixel coordinates, and pixel coordinates-based metrics (also referred to as absolute metrics) may be derived from said distributions, including one or more of an absolute overlap (PRo′) between the pain drawing and the response map, an absolute coverage (PRc′), or an absolute selectivity (PRs′). The PRo′, PRc′, and PRs′ may be computed using Equations (8)-(10), respectively.

In some examples, the PTM, or the PRo, may be determined using one or more of the CM, dMax, and Φ respectively computed for the pain distribution across the dermatomal compartment set {d_{P}}, and for the response distribution across the dermatomal compartment set {d_{R}}. By way of example and not limitation, the PTM may be determined using a linear or nonlinear combination of differences of CM, dMax, and Φ for the pain distribution and the response distribution, such as given in Equation (11).

At 2550, an optimal stimulation setting may be determined based at least on one or more of the PTMs obtained at 2540, such as via the electrode configuration circuit 2032. The stimulation setting, also referred to as stimulation field definition, includes stimulation parameters such as field size, shape, and intensity, field scaling and steering parameters, among others. In an example, the PTM may be used to determine for each electrode a corresponding anodic weight and a cathodic weight. The electrode has a net anode effect if the anodic weight is greater than the cathodic weight, or a net cathode effect if the anodic weight is less than the cathodic weight. The anodic and cathodic weights may be used to determine a target stimulation field definition, which defines size, shape, and field intensity, as well as scaling and steering of the field. The target stimulation field definition may be mapped to electrode polarities and current fractionalization for the electrodes on the stimulation lead. In an example, the mapping involves application of a transfer matrix to the target stimulation field definition. The transfer matrix may be used to compute relative strengths of a plurality of constituent current sources needed to match the target stimulation field definition at the spatial observation points when a specified optimization criterion is satisfied. In another example, the mapping involves a regression fitting of the target stimulation field definition, such as by using a linear or nonlinear regression model. In an example, the mapping may involve a least-square fitting of the target electrical field.

The optimal stimulation setting and the fractionalized electrode configuration may be output to a user or a process at 2560. In an example, at 2562, the fractionalized electrode configuration may be programmed to a neuromodulation device, such as via a programming device or the stimulation controller 2033. The programming of the neuromodulation device may be carried out automatically or triggered by a user command or a specific event. A programming control signal may be communicated to the neuromodulation device via a communication link such as the wireless communication network. In response to the control signal, the neuromodulation device may deliver neuromodulation energy, such as sub-perception stimulation energy, at the target neural element (e.g., spinal cord or affiliated neural structures) in accordance with the fractionalized electrode configuration to relieve pain. In another example, at 2564, the optimal stimulation field and the fractionalized electrode configuration, among other information such as the pain distribution, the response distribution (e.g., paresthesia distribution or sensor data distribution), the dermatomal metrics derived from pain distribution and the response distribution, or various PTMs, may be presented to a system user (e.g., a clinician), such as displayed on a user interface of the output unit 2050, or the GUI 214 or 414 of the programming device 213, 413, or 628. The user may adjust the stimulation parameter through said user interface. In yet another example, at 2566, the fractionalized electrode configuration, among other intermediate computations such as dermatomal metrics and various PTMs, may be stored in a storage device for future use.

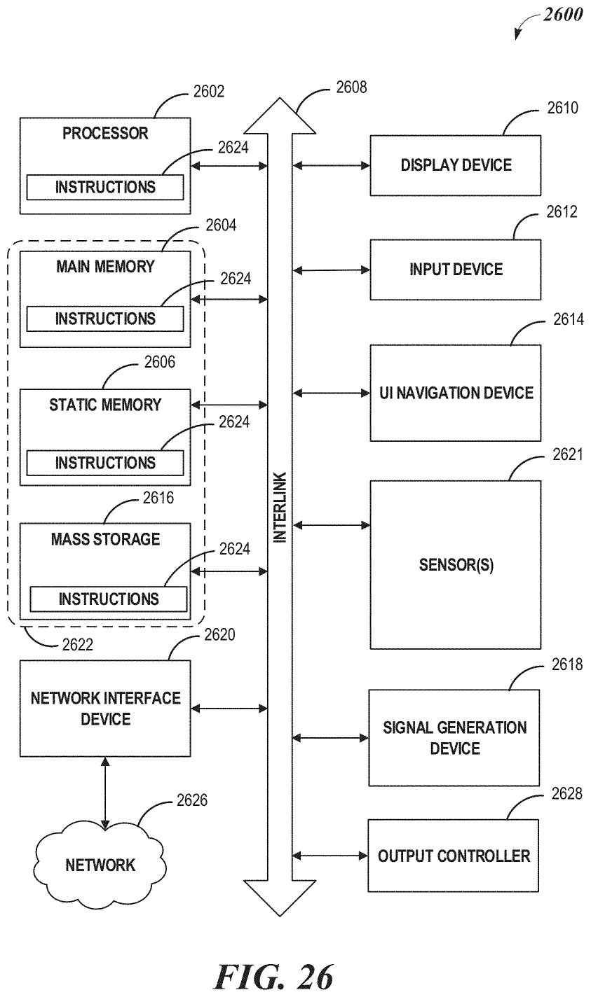

FIG. 26 illustrates generally a block diagram of an example machine 2600 upon which any one or more of the techniques (e.g., methodologies) discussed herein may perform. Portions of this description may apply to the computing framework of various portions of the neuromodulation device or the external programming device.

In alternative embodiments, the machine 2600 may operate as a standalone device or may be connected (e.g., networked) to other machines. In a networked deployment, the machine 2600 may operate in the capacity of a server machine, a client machine, or both in server-client network environments. In an example, the machine 2600 may act as a peer machine in peer-to-peer (P2P) (or other distributed) network environment. The machine 2600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 2600 may include a hardware processor 2602 (e.g., a central processing unit (CPU), a graphics processing unit (GPU), a hardware processor core, or any combination thereof), a main memory 2604 and a static memory 2606, some or all of which may communicate with each other via an interlink (e.g., bus) 2608. The machine 2600 may further include a display unit 2610 (e.g., a raster display, vector display, holographic display, etc.), an alphanumeric input device 2612 (e.g., a keyboard), and a user interface (UI) navigation device 2614 (e.g., a mouse). In an example, the display unit 2610, input device 2612 and UI navigation device 2614 may be a touch screen display. The machine 2600 may additionally include a storage device (e.g., drive unit) 2616, a signal generation device 2618 (e.g., a speaker), a network interface device 2620, and one or more sensors 2621, such as a global positioning system (GPS) sensor, compass, accelerometer, or other sensor. The machine 2600 may include an output controller 2628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 2616 may include a machine readable medium 2622 on which is stored one or more sets of data structures or instructions 2624 (e.g., software) embodying or utilized by any one or more of the techniques or functions described herein. The instructions 2624 may also reside, completely or at least partially, within the main memory 2604, within static memory 2606, or within the hardware processor 2602 during execution thereof by the machine 2600. In an example, one or any combination of the hardware processor 2602, the main memory 2604, the static memory 2606, or the storage device 2616 may constitute machine readable media.

While the machine readable medium 2622 is illustrated as a single medium, the term “machine readable medium” may include a single medium or multiple media (e.g., a centralized or distributed database, and/or associated caches and servers) configured to store the one or more instructions 2624.

The term “machine readable medium” may include any medium that is capable of storing, encoding, or carrying instructions for execution by the machine 2600 and that cause the machine 2600 to perform any one or more of the techniques of the present disclosure, or that is capable of storing; encoding or carrying data structures used by or associated with such instructions. Non-limiting machine readable medium examples may include solid-state memories, and optical and magnetic media. In an example, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 2624 may further be transmitted or received over a communications network 2626 using a transmission medium via the network interface device 2620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 2620 may include one or more physical jacks (e.g., Ethernet, coaxial, or phone jacks) or one or more antennas to connect to the communications network 2626. In an example, the network interface device 2620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 2600, and includes digital or analog communications signals or other intangible medium to facilitate communication of such software.

Various embodiments are illustrated in the figures above. One or more features from one or more of these embodiments may be combined to form other embodiments.

The method examples described herein may be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code may form portions of computer program products. Further, the code may be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system for controlling neuromodulation therapy for pain relief in a patient, the system comprising:
an electrostimulator configured to apply first neuromodulation energy to a target neural element of the patient according to a stimulation setting;
a data receiver configured to receive pain data including information of a pain site on a body of the patient, and to receive physiological data including information of a body site responsive to the applied first neuromodulation energy;
a processor circuit configured to:
determine a pain distribution across a first set of dermatomal compartments using the received pain data, and determine a response distribution across a second set of dermatomal compartments using the received physiological data;
generate a pain targeting metric (PTM) using the pain distribution and the response distribution, the PTM representing a spatial correspondence between the pain site and the body site responsive to the applied first neuromodulation energy at one or more dermatomal compartments; and
determine an optimal stimulation setting for neuromodulation pain therapy using the generated PTM.

2. The system of claim 1, wherein the electrostimulator is configured to apply second neuromodulation energy to the target neural element for pain relief in accordance with the optimal stimulation setting, the second neuromodulation energy different from the first neuromodulation energy.

3. The system of claim 1, wherein the received pain data includes a pain drawing of the pain site associated with a pain score, and the received physiological data includes a response map of the body site responsive to the applied first neuromodulation energy, the body site associated with a response score.

4. The system of claim 3, wherein the response map includes a paresthesia drawing of a body site experiencing paresthesia in response to the applied first neuromodulation energy.

5. The system of claim 3, wherein the response map includes a sensor data map including sensor data collected at a body site in response to the applied first neuromodulation energy, the sensor data including one or more of:
electromyography (EMG) data;
electrospinogram (ESG) data;
electrically evoked compound action potential (eCAP) data; or
impedance data.

6. The system of claim 3, wherein the response map includes sensor data collected at a body site in response to the applied first neuromodulation energy, the sensor data including one or more of:
photoplethysmography (PPG) data;
near-infrared spectroscopy (NIRS) data;
doppler flowmetry data;
accelerometer sensor data; or
gyroscope sensor data.

7. The system of claim 3, wherein the processor circuit is configured to:
pixelate the pain drawing into pixels corresponding to anatomical point locations of the pain site, determine for each of the first set of dermatomal compartments a respective dermatome-level pain effect using the pixelated pain drawing, and generate the pain distribution using the dermatome-level pain effects of the first set of dermatomal compartments;
pixelate the response map into pixels corresponding to anatomical point locations of the body site responsive to the applied first neuromodulation energy, determine for each of the second set of dermatomal compartments a respective dermatome-level physiological response using the pixelated response map, and generate the response distribution using the dermatome-level physiological responses of the second set of dermatomal compartments.

8. The system of claim 7, wherein the processor circuit is configured to:
generate a first dermatomal metric from the pain distribution, and generate a second dermatomal metric from the response distribution; and
generate the PTM using the first and second dermatomal metrics.

9. The system of claim 8, wherein the first dermatomal metric includes a center of mass of the pixelated pain drawing based on the dermatome-level pain effects of the first set of dermatomal compartments, and wherein the second dermatomal metric includes a center of mass of the pixelated response map based on the dermatome-level physiological responses of the second set of dermatomal compartments.

10. The system of claim 8, wherein the first dermatomal metric includes a peak dermatome representing a dermatomal compartment having a largest dermatome-level pain effect among the first set of dermatomal compartments, and wherein the second dermatomal metric includes a peak dermatome representing a dermatomal compartment having a largest dermatome-level physiological response among the second set of dermatomal compartments.

11. The system of claim 8, wherein the first dermatomal metric includes a dermatomal spread representing a subset of the first set of dermatomal compartments that have a dominant dermatome-level pain effect, and wherein the second dermatomal metric includes a dermatomal spread representing a subset of the second set of dermatomal compartments that have a dominant dermatome-level physiological response.

12. The system of claim 7, wherein the processor circuit is configured to generate the PTM using one or more of:
an overlap between the pixelated pain drawing and the pixelated response map, the overlap based on the dermatome-level pain effects and the dermatome-level physiological responses across a union of the first and second sets of dermatomal compartments;
a ratio of the overlap to a sum of the dermatome-level pain effects across the first set of dermatomal compartments; or
a ratio of the overlap to a sum of the dermatome-level physiological responses across the second set of dermatomal compartments.

13. The system of claim 1, wherein the processor circuit is configured to:
evaluate the PTM for each of a plurality of candidate stimulation settings; and
select from the plurality of candidate stimulation settings an optimal stimulation setting with a corresponding PTM satisfying a specific condition.

14. The system of claim 1, comprising a user interface configured to display one of more of the pain distribution, the response distribution, or the PTM, and to receive a user input for adjusting one or more stimulation parameters including:
a stimulation electrode position;
a stimulation pulse width;
a stimulation amplitude;
a stimulation rate; or
a stimulation pulse waveform.

15. A method for controlling neuromodulation therapy for pain relief in a patient, the method comprising:
receiving pain data including information of a pain site on a body of the patient;
applying first neuromodulation energy to a target neural element of the patient according to a stimulation setting;
receiving physiological data including information of a body site responsive to the applied first neuromodulation energy;
determining a pain distribution across a first set of dermatomal compartments using the received pain data, and determining a response distribution across a second set of dermatomal compartments using the received physiological data;
generate a pain targeting metric (PTM) using the pain distribution and the response distribution, the PTM representing a spatial correspondence between the pain site and the body site responsive to the applied first neuromodulation energy at one or more dermatomal compartments; and
determining an optimal stimulation setting for neuromodulation pain therapy using the generated PTM.

16. The method of claim 15, wherein the received pain data includes a pain drawing and the received physiologic data includes a response map, and
wherein determining the pain distribution includes steps of: pixelating the pain drawing; determining for each of the first set of dermatomal compartments a respective dermatome-level pain effect using the pixelated pain drawing; and generating the pain distribution using the dermatome-level pain effects of the first set of dermatomal compartments; and
wherein determining the response distribution includes steps of: pixelating the response map; determining for each of the second set of dermatomal compartments a respective dermatome-level physiological response using the pixelated response map; and generating the response distribution using the dermatome-level physiological responses of the second set of dermatomal compartments.

17. The method of claim 16, wherein generating the PTM includes using a comparison between a first dermatomal metric derived from the pain distribution and a second dermatomal metric derived from the response distribution.

18. The method of claim 17, wherein:
the first dermatomal metric includes a center of mass of the pixelated pain drawing based on the dermatome-level pain effects of the first set of dermatomal compartments, and the second dermatomal metric includes a center of mass of the pixelated response map based on the dermatome-level physiological responses of the second set of dermatomal compartments;
the first dermatomal metric includes a peak dermatome representing a dermatomal compartment having a largest dermatome-level pain effect among the first set of dermatomal compartments, and the second dermatomal metric includes a peak dermatome representing a dermatomal compartment having a largest dermatome-level physiological response among the second set of dermatomal compartments; or
the first dermatomal metric includes a dermatomal spread representing a subset of the first set of dermatomal compartments that have a dominant dermatome-level pain effect, and the second dermatomal metric includes a dermatomal spread representing a subset of the second set of dermatomal compartments that have a dominant dermatome-level physiological response.

19. The method of claim 16, wherein generating the PTM includes using at least one of:
an overlap between the pixelated pain drawing and the pixelated response map, the overlap based on the dermatome-level pain effects and the dermatome-level physiological responses across a union of the first and second sets of dermatomal compartments;
a ratio of the overlap to a sum of the dermatome-level pain effects across the first set of dermatomal compartments; or
a ratio of the overlap to a sum of the dermatome-level physiological responses across the second set of dermatomal compartments.

20. The method of claim 15, comprising:
evaluating the PTM for each of a plurality of candidate stimulation settings; and
selecting from the plurality of candidate stimulation settings an optimal stimulation setting with a corresponding PTM satisfying a specific condition.
